(19) United States
(12) Patent Application Publication
(10) Pub. No.: US 2007/0010816 A1
(43) Pub. Date: Jan. 11, 2007

(54) Tool driver, coupler and associated method

(76) Inventors: Trent R. Wilkinson, Larwill, IN (US); Anthony J. Metzinger, Winona Lake, IN (US); Michael Tucker, Augusta, GA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/170,568

(22) Filed: Jun. 29, 2005

(51) Int. Cl. A61F 2/30

(52) U.S. Cl. 606/69

(57) ABSTRACT

A tool driver for use in orthopaedics to install an implant into bone with a power source is provided. The tool driver includes an expandable connector cooperable with the implant for holding the implant to the expandable connector. The tool driver also includes a drive connector for connecting the tool driver to the power source. The tool driver further includes an actuator operably connected to the expandable connector for actuating the expandable connector. The actuator is at least partially connected to the drive connector while the actuator actuates the expandable connector.

### CROSS-REFERENCE TO RELATED APPLICATIONS

Cross-reference is made to the following application: DEP 5420USNP2 titled “SCREWDRIVER, KIT AND ASSOCIATED METHOD” filed concurrently herewith which is incorporated herein by reference.

### TECHNICAL FIELD OF THE INVENTION

The present invention relates to devices for applying a torque to an orthopaedic implant component and, more particularly, to a driver grasping the orthopaedic implant component while applying a torque to an orthopaedic implant component.

### BACKGROUND INFORMATION

A joint within the human body forms a juncture between two or more bones or other skeletal parts. The ankle, hip, knee, shoulder, elbow and wrist are just a few examples of the multitude of joints found within the body. As should be apparent from the above list of examples of joints, many of the joints permit relative motion between the bones. For example, the motion of sliding, gliding, and hinge or ball and socket movements may be had by a joint. For example, the ankle permits a hinge movement, the knee allows for a combination of gliding and hinge movements and the shoulder and hip permit movement through a ball and socket arrangement.

The joints in the body are stressed or can be damaged in a variety of ways. For example, the gradual wear and tear is imposed on the joints through the continuous use of a joint over the years. The joints that permit motion have cartilage positioned between the bones providing lubrication to the motion and also absorbing some of the forces direct to the joint. Over time, the normal use of a joint may wear down the cartilage and bring the moving bones in a direct contact with each other. In contrast, in normal use, a trauma to a joint, such as the delivery of a large force, from an accident for, example, an automobile accident, may cause considerable damage to the bones, the cartilage or to other connective tissue such as tendons or ligaments.

Arthropathy, a term referring to a disease of the joint, is another way in which a joint may become damaged. Perhaps the known joint disease is arthritis, which is generally referred to a disease or inflammation of a joint that results in pain, swelling, stiffness, instability, and often deformity.

There are many different forms of arthritis, with osteoarthritis being the most common and resulting from the wear and tear of a cartilage within a joint. Another type of arthritis is osteonecrosis, which is caused by the death of a part of the bone due to loss of blood supply. Other types of arthritis are caused by trauma to the joint while others, such as rheumatoid arthritis, Lupus, and psoriatic arthritis destroy cartilage and are associated with the inflammation of the joint lining.

The hip joint is one of the joints that are commonly afflicted with arthropathy. The hip joint is a ball and socket joint that joins the femur or thighbone with the pelvis. The pelvis has a semispherical socket called the acetabulum for receiving a ball socket head in the femur. Both the head of the femur and the acetabulum are coated with cartilage for allowing the femur to move easily within the pelvis. Other joints commonly afflicted with arthropathy include the spine, knee, shoulder, carpals, metacarpals, and phalanges of the hand.

Arthroplasty as opposed to arthropathy commonly refers to the making of a artificial joint. In severe cases of arthritis or other forms of arthropathy, such as when pain is overwhelming or when a joint has a limited range of mobility, a partial or total replacement of the joint within an artificial joint may be justified. The procedure for replacing the joint varies, of course, with the particular joint in question, but in general involves replacing a terminal portion of an afflicted bone with a prosthetic implant and inserting a member to serve as a substitute for the cartilage.

The prosthetic implant is formed of a rigid material that becomes bonded with the bone and provides strength and rigidity to the joint and the cartilage substitute members chosen to provide lubrication to the joint and to absorb some of the compressive forces. Suitable materials for the implant include metals, and composite materials such as titanium, cobalt chromium, stainless steel, ceramic and suitable materials for cartilage substitutes include polyethylene. A cement may also be used to secure the prosthetic implant to the host bone.

The long bones including the femur, fibula, tibia, humerus, radius and ulna are in addition to the effects of osteoarthritis to their joints are particularly exposed to trauma from accident. As such they often are fractured during such trauma and may be subject to complex devastating fractures.

Automobile accidents, for instance, are a common cause of trauma to long bones. In particular, the femur and tibia frequently fracture when the area around the knee is subjected to a frontal automobile accident.

Often the distal end or proximal portions of the long bone, for example the femur and the tibia, are fractured into several components and must be realigned. Mechanical devices, commonly in the forms of pins, plates, screws, nails, wires and external devices are commonly used to attach fractured long bones. The pins, plates, wires, nails and screws are typically made of a durable material compatible to the human body, for example titanium, stainless steel or cobalt chromium.

Fractures of the long bone are typically secured into position by at least one of three possible techniques.

The first method is the use of intramedullary nails that are positioned in the intramedullary canal of those portions of the fractured bone.

A second method of repairing fractured bones is the use of internal bone plates that are positioned under the soft tissue and on the exterior of the bone and bridges the fractured portion of the bone.

Various types of orthopaedic implants such as spine implants, trauma plates, rods and other devices, as well as, joint prosthetics typically utilize and/or rely on components that must be securely attached to other components of the implant or to various parts of the body. The integrity and/or effectiveness of the implant may depend upon proper attachment of the component. Particularly, if the component is either over-tightened or under-tightened, there can be associated negative effects. For example, an under-tightened component may loosen causing the loss of effectiveness of a component, while an over-tightened component may impart an undesirable amount of stress on one or more components.

Implants are thus attached using devices that will allow the surgeon to apply the necessary torque throughout the attachment process, since a certain level of torque is required to properly secure a component. It is often difficult, however, to ascertain when the proper level of torque has been imparted on an implant component and, in turn, when the implant component has been securely attached.

In order to alleviate these problems, torque-limiting devices or drivers have been developed to help ensure that a consistent or limited assembly torque is imparted on implant components in order to properly secure torque-applied implant components to other implant components and/or body parts. Torque-limiting drivers are calibrated to impart a desired level of torque to an implant component during implant thereof. Other torque-limiting drivers offer user adjustable calibration for varying the level of applied torque.

The present invention is directed to alleviate at least some of the aforementioned problems.

Fasteners, for example screws and pins are utilized to secure orthopaedic implants in the form of plates and nails, as well as joint prosthesis, to adjoining bone. Drivers are typically used to secure the screws and pins to the bone. The driver may include a power driver feature. For example, a power tool in the form of an pneumatic, hydraulic or electrical, for example a battery driven electrical driver may be used. It is helpful for the screw or pin to securely fasten to the locking driver. It is also beneficial for the driver to have the capability of being hand driven for perhaps a portion of the insertion of the screw or pin.

Procedures for implanting the orthopaedic implants including, for example, orthopaedic trauma, intermedullary nails and orthopaedic bone plates as well as for orthopaedic implants are becoming more advanced and precise. After an implant is implanted into the bone, screws and or pins may be driven through openings in the implant to hold the implant in place. To obtain correct alignment of the screw or pin within or to the orthopaedic implant, a jig or fixture, which outlines the correct screw or pin position, may be attached to the implant.

Sheaths are often used in conjunction with the jig or fixture to ensure the proper alignment of the instrumentation and proper placement of the pins and screws. Often the bone where the pin or screw is to be inserted is pre-drilled to form a hole for later insertion of the screw or pin. Once the hole is drilled, the screw or pin may then be passed through a sheath and driven into the bone.

Because the screw has to be driven through a sheath, the screw can easily fall off the driver and cause problems with the procedure.

Attempts have been made to solve the problem of screws and pins separating from the driver, for example, special drivers have been created which lock the screw onto the driver. In this fashion, the screw or pin will remain fixed to the driver until the screw is properly driven into the bone. Although these drivers are somewhat successful to hold the screws, most of them are hand-operated instruments.

A few power-driven instruments incorporate a locking feature strong enough to hold screws during an implant implantation procedure have been provided.

For example, Smith & Nephew, Memphis, Tenn., provides a special power driver and screw, which mate and lock together. This special power driver is more fully described in U.S. Pat. No. 6,565,573 incorporated herein in its entirety by reference. The screws have internal threads within a hexagonal recess. The driver has a threaded stud, which mates with the internal threads in the screw. The threaded stud passes coaxially through a hexagonal driver, which mates with the hexagonal recess in the screw. The threaded stud locks the screw onto the driver by engaging with the internal threads of the screw.

This system works well to hold the screws, but is not easy to use. To lock the driver onto the screw, the driver must be removed from the power instrument. After the driver is removed, the threaded stud can be engaged with the screw and locked into place. Subsequently, the driver can be inserted into the power instrument and the screw driven into place. Again, to unlock the screw, the driver must be removed from the power instrument. Also, this driver works only with screws that have the necessary internal threads.

### SUMMARY

The present invention is directed toward a coupling system for use with a power-driven locking driver. The driver is used to securely hold screws and drive them into bone. The coupling system allows the driver to operate the locking feature of the driver and transmit torque from the power instrument to the driver. The locking driver of the present invention may have three features. The first feature is an attachment feature and the second feature is a locking mechanism to lock the screw. The third feature is a coupling system.

The attachment feature for use with the locking driver of the present invention can be of any standard configuration used in power tools. Such attachment features are known as the AO system, available from Synthes, Inc. West Chester, Pa. 19380 or the Hudson System available from Hudson Surgical, Inc.

The locking driver of the present invention further includes a locking mechanism. The locking mechanism may utilize a colleted screw holder. A colleted screw holder is shown in U.S. Pat. No. 6,286,401 to Hanjipour and assigned to the same assignee as the present invention. The Solid Lock Screw Driver incorporates a locking mechanism as described in U.S. Pat. No. 6,286,401. The Solid Lock Screw Driver is used in the DePuy Versa Nail Set.

The locking driver of the present invention further includes the coupling system. The coupling system marries the attachment feature and the locking mechanism in such a way that the user can operate the locking mechanism without removing the driver from the power instrument. To lock the screw onto the driver, the user rotates for example, clockwise the instrument attachment into the main body.

The instrument is attached onto the main body with the use of mating internal and external threads. In order to rotate for the instrument attachment, with respect to the main body, the collar is slid away from the instrument attachment. The user then threads the instrument attachment into the main body until the screw is locked onto the driver. The collar is then released and the spring pushes the collar back toward the instrument attachment.

The collar may, for example have a twelve-point inner-periphery which mates with the hexagonal outer periphery of the instrument attachment and of the main body. When the collar is mating with both the instrument attachment and the main body, the driver can be used to drive the screw into place. To unlock the screw, the collar is again slid away from the instrument attachment. The instrument attachment is then rotated counter-clockwise and the screw is released. The entire coupling can be accomplished without removing the instrument from the power driver.

According to one aspect of the invention a tool driver for use in orthopaedics to install an implant into bone with a power source is provided. The tool driver includes an expandable connector cooperable with the implant for holding the implant to the expandable connector. The tool driver also includes a drive connector for connecting the tool driver to a power source. The tool driver further includes an actuator operably connected to the expandable connector for actuating the expandable connector. The actuator is at least partially connected to the drive connector while the actuator actuates the expandable connector.

According to another aspect of the invention a coupler for use with a tool driver and a tool holder for use in orthopaedics to install a tool into bone with a power source is provided. The coupler includes a member operably associated with the implant holder and with the tool driver. The member has a first relationship with the implant holder and the tool driver in which the implant holder and the tool driver are connected and a second relationship with the implant holder and the tool driver in which the implant holder and the tool driver are at least partially disconnected.

According to another aspect of the invention a screwdriver for use in orthopaedics to install a screw into bone with a power tool is provided. The screwdriver includes an expandable connector cooperable with the screw for holding the screw to the expandable connector. The screwdriver also includes a drive connector for connecting the screwdriver to the power tool and an actuator. The actuator is operably connected to the expandable connector for actuating the expandable connector. The actuator is at least partially connected to the drive connector while the actuator actuates the expandable connector.

According to another aspect of the invention a kit for use in orthopaedics in installing a screw into bone is provided. The kit includes a power tool and a screwdriver. The screwdriver is for selective expandable engagement with the screw. The screwdriver includes a drive connector for connecting the screwdriver to the power tool and an actuator operably connected to the expandable connector for actuating the expandable connector. The actuator is at least partially connected to the drive connector while the actuator actuates the expandable connector.

According to another aspect of the invention a method for performing orthopaedic surgery on a bone is provided. The method includes the steps of providing a screw for attachment to the bone and providing a kit for installing the screw into the bone. The kit includes a power tool and a screwdriver for selective expandable engagement with the screw. The screwdriver includes a drive connector for connecting the screwdriver to the power tool and an actuator operably connected to the expandable connector for actuating the expandable connector. The actuator at least partially connected to the drive connector while the actuator actuates the expandable connector. The method also includes the steps of connecting the screw to the screwdriver while the screwdriver is at least partially operatively disconnected from the power tool and operatively connecting the power tool to the screwdriver. The method also includes the step of securing the screw to the bone using the power tool and the screwdriver.

According to yet another aspect of the invention a method for performing orthopaedic surgery on a bone is provided. The method includes the steps of providing a screw for attachment to the bone and providing a kit for installing the screw into the bone. The kit includes a power tool and a screwdriver for engagement with the screw. The screwdriver includes a drive connector for connecting the screwdriver to the power tool and a coupler operably associated with the implant holder and with the tool driver. The coupler has a first relationship with the implant holder and the tool driver in which the implant holder and the tool driver are rotatably connected and a second relationship with the implant holder and the tool driver in which the implant holder and the tool driver are rotatably disconnected.

The method also includes the step of hand tightening the screw to the screwdriver while the screwdriver is rotatably disconnected from the power tool.

The method also includes the steps of operatively connecting the power tool to the screwdriver and securing the screw to the bone using the power tool and the screwdriver.

The technical advantages of the present invention include the ability to lock a screw onto and unlock a screw from the screwdriver without removing the driver from the power instrument. For example, according to one aspect of the present invention, a tool driver for use in orthopaedic to install an implant into a bone with a power source is provided.

The tool driver includes an expandable connector cooperable with the implant, for example a bone screw, for holding the bone screw to the expandable connector. The tool driver also includes a drive connector for connecting the tool driver to the power source. The tool driver further includes an actuator operably connected to the expandable connector for actuating the expandable connector.

The actuator is at least partially connected to the drive connector while the actuator actuates the expandable connector. Thus, the present invention provides the ability to lock and to unlock the screw from the driver without the removal of the driver from the power instrument.

The technical advantages of the present invention further include the ability to hand-tighten an orthopaedic screw without using a power tool. For example, according to another aspect of the present invention a coupler is provided for use with a tool driver and an implant holder for use in orthopaedics to install an implant, for example an orthopaedic screw, into bone with a power source.

The coupler includes a member that is operably associated with the orthopaedic screw holder and with the tool driver. The member has a first relationship with the orthopaedic screw holder and the tool driver in which the orthopaedic screw holder and the tool driver are connected. The member further has a second relationship with the screw holder and the tool driver in which the screw holder and the tool driver are at least partially disconnected. While the screw holder and the tool driver are disconnected, the operator may rotate the member by hand to hand heighten the screw. Thus, the present invention provides for the ability to hand-tighten or use a power tool with a common device.

The technical advantages of the present invention further include the ability to pre-tighten an orthopaedic screw by hand and then subsequently tighten it with a power tool. For example, according to yet another aspect of the present invention, a coupler is provided for use with a tool driver and a screw holder for use in orthopaedics to install an orthopaedic screw into bone with a power source. The coupler includes a member associated with the screw holder and with the tool driver.

The member has a first relationship with the screw holder and the tool driver in which the screw holder and the tool driver are connected as well as a second relationship with the screw holder and the tool driver in which the screw holder and the tool driver are partially disconnected. While the screw holder and the tool driver are disconnected, the operator can pre-tighten by hand the screw with the coupler and then, after the screw has been hand-tightened, the screw holder and the tool driver's relationship can be modified such that the screw can be tightened with the power source.

The technical advantages of the present invention further include the ability to easily clean and sterilize an orthopaedic implant to a holder and driver. For example, according to another aspect of the present invention, a screwdriver for use in orthopaedics to install a screw into bone with a power tool is provided. The screwdriver includes an expandable connector removably cooperable with the screw for holding the screw to the expandable connector. A tool driver for connecting the screwdriver to the power tool is provided which is removably connected to the expandable connector. The screwdriver further includes an actuator operably connectable and removable from the expandable connector for actuating the expandable connector. The actuator is at least partially connected to the drive connector when the actuator actuates the expandable connector.

Other technical advantages of the present invention will be readily apparent to one skilled in the art from the following figures, descriptions and claims.

### BRIEF DESCRIPTION OF THE DRAWINGS

In the drawings:

FIG. 1 is a plan view of a locking power driver in accordance with an embodiment of the present invention;

FIG. 1A is a plan view of various implants with which the power locking driver of FIG. 1 may cooperate;;

FIG. 2 is an cross section view of the locking power driver of FIG. 1 along the line 2-2 in the direction of the arrows;

FIG. 3 is a partial enlarged plan view of the locking power driver of FIG. 1 without the power locking mechanism;

FIG. 4 is a plan view of the collet and the actuator of the locking power driver of FIG. 1;

FIG. 4A is a partial plan view of the flexible member of the power driver of FIG. 1;

FIG. 4B is a partial plan view of another embodiment of the present invention in the form of a power driver having a collet with double slits;

FIG. 5 is a plan view of the locking power driver of FIG. 1 shown in the locked position;

FIG. 6 is a plan view of the locking power driver of FIG. 1 shown in the un-locked position;

FIG. 7 is a plan view of the locking power driver of FIG. 1 shown in an un-locked extended position;

FIG. 7A is a plan view of the expandable member of the locking driver of FIG. 1 and a larger expandable member for use in securing a larger screw for use in the locking driver of FIG. 1;

FIG. 8 is a plan view of the locking power driver of FIG. 1 showing the sleeve, adaptor and spring in greater detail;

FIG. 9 is a plan view of the flexor and body of the locking power driver of FIG. 1;

FIG. 10 is a plan view of the flexible member of the locking power driver of FIG. 1;

FIG. 11 is a plan view of the components that comprise the locking power driver of FIG. 1;

FIG. 11A is a plan view of modular components for the locking driver of the present invention;

FIG. 12 is a plan view of a handle for use with the locking power driver of FIG. 1;

FIG. 13 is a plan view of the spring for use with the locking power driver of FIG. 1;

FIG. 14 is a perspective view of the collar of the locking power driver of FIG. 1;

FIG. 14A is a top view of another collar with hexagonal splines for use with the locking power driver of FIG. 1;

FIG. 15 is a plan view of the collar of FIG. 14;

FIG. 16 is a cross sectional view of the collar of FIG. 15 taken along line 16-16 thereof in the direction of the arrows;

FIG. 17 is an end view of the collar of FIG. 15;

FIG. 18 is a plan view partially in cross-section of the flexible member of the locking power driver of FIG. 1;

FIG. 19 is a partial plan view of the flexible member of FIG. 18;

FIG. 20 is a plan view of the flexor of the locking power driver of FIG. 1;

FIG. 21 is a plan view of the flexible member of the locking power driver of FIG. 1;

FIG. 22 is a cross sectional view of the flexible member of FIG. 21 taken along line 22-22 thereof in the direction of the arrows;

FIG. 23 is a perspective view of the nut of the locking power driver of FIG. 1;

FIG. 24 is a plan view of the nut of FIG. 23;

FIG. 25 is a cross-sectional view of FIG. 24 along the line 25-25 in the direction of the arrows;

FIG. 26 is a top view of the nut of FIG. 24;

FIG. 27 is a perspective view of the body of the locking power driver of FIG. 1;

FIG. 28 is a plan view of the flexible member of the power driver of FIG. 1 shown in a spaced-apart relationship with the screw as well as in contact with the screw;

FIG. 29 is a plan view of a collet for use with the flexible member of FIG. 28;

FIG. 29A is a plan view of another collet for use with the flexible member of FIG. 28;

FIG. 30 is a plan view of a locking power driver in accordance with another embodiment of the present invention;

FIG. 30A is a partial plan view of the locking power driver of FIG. 30 with a different tip;

FIG. 31 is a plan view of a locking power driver in accordance with yet another embodiment of the present invention;

FIG. 32 is a plan view of a locking power driver in accordance with a further embodiment of the present invention;

FIG. 33 is a plan view of a locking power driver in accordance with another embodiment of the present invention;

FIG. 33A is a partial plan view partially in cross-section of FIG. 33 showing the taper lock in greater detail;

FIG. 33B is a partial plan view partially in cross-section of FIG. 33;

FIG. 34 is a plan view of a locking power driver in accordance with a further embodiment, of the present invention;

FIG. 34A is a partial plan view of FIG. 34 partially in cross-section;

FIG. 35 is a plan view of a locking power driver in accordance with yet another embodiment of the present invention;

FIG. 35A is a partial plan view of FIG. 35 partially in cross-section;

FIG. 36 is a plan view of a locking power driver in accordance with another embodiment of the present invention;

FIG. 37 is a partial plan view of a locking power driver in accordance with yet another embodiment of the present invention;

FIG. 38 is a partial plan view of a kit in accordance with a further embodiment of the present invention;

FIG. 39 is a flow diagram of a method of performing surgery in accordance with yet another embodiment of the present; and

FIG. 40 is a flow diagram of another method of performing surgery in accordance with another embodiment of the present invention.

Corresponding reference characters indicate corresponding parts throughout the several views. Like reference characters tend to indicate like parts throughout the several views.

### DESCRIPTION OF THE PREFERRED EMBODIMENT(S)

While the invention is susceptible to various modifications and alternative forms, specific embodiments thereof have been shown by way of example in the drawings and will herein be described in detail. It should be understood, however, that there is no intent to limit the invention to the particular forms disclosed, but on the contrary, the intention is to cover all modifications, equivalents, and alternatives falling within the spirit and scope of the invention.

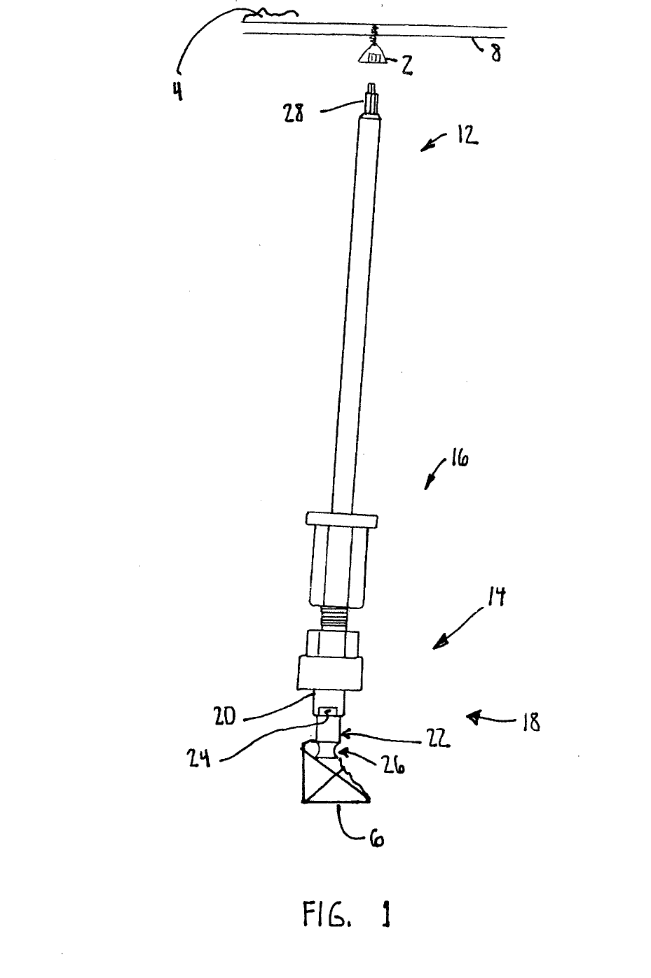

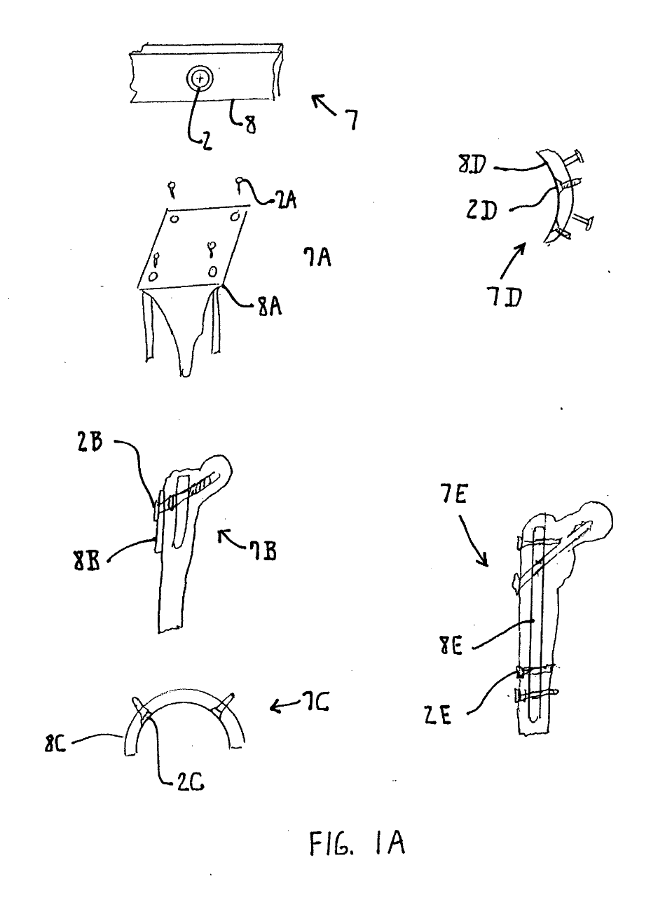

Referring now to FIG. 1, an embodiment of the present invention is shown as tool driver 10. The tool driver 10 is designed for use in orthopaedics to install an implant 2, for example and as shown in FIG. 1, a screw into bone 4 with a power source 6. The bone 4 as shown in FIG. 1 may be in the-form of a long bone, for example a femur.

The power source 6 may be any commercially available power source for use to rotate a tool during orthopaedic surgery. For example, the power source 6 may be in the form of a power drill. The power drill may be one of many types. For example, the power drill may be in the form of a pneumatic power drill, a hydraulic power drill, or an electric power drill. If an electric power drill, the power drill may be in the form of a battery powered power drill.

The implant 2 may be in the form of a screw that is positioned independently into bone 4. Alternatively, and as shown in FIG. 1, the implant 2 in the form of the screw 2 may be implanted in connection with another, perhaps larger, implant in the form of, for example, a bone plate. It should be appreciated that additional spaced apart implants in the form of additional screws (not shown) may be installed with the tool driver 10 of the present invention.

As shown in FIG. 1, the tool driver 10 includes an expandable connector 12, which is cooperable with the screw 2 for holding the screw 2 to the expandable connector 12. The tool driver 10 further includes a drive connector 14 for connecting the tool driver 10 to the power source 6.

The tool driver 10 further includes an actuator 16, which is operably connected to the expandable connector 12. The actuator 16 is utilized for actuating the expandable connector 12. The actuator 16 may, as shown in FIG. 1 be partially or completely connected to the drive connector 14 while the actuator 16 is utilized to actuate the expandable connector 12.

The expandable connector 12 may have any suitable form capable of expandably containing an implant, for example screw 2. The drive connector 14 may include a drive adaptor 18 including, for example, a cylindrical shaped base 20 and a cylindrical shaped stem 22 extending from the base 20. A series of flats 24, for example a pair of opposed flats 24, may be formed on the base 20. A groove 26 in the form of a circumferential groove may be formed on the stem 20.

The drive adapter 18 may, for example, be a drive adapter that is commercially available for connection with commercially available power equipment. Such commercially available connectors are in the form of quick disconnectors known as the AO System, available from Synthes, Inc. West Chester, Pa., 19380. Alternatively, the drive adapter 18 may be in the form of a Hudson Adapter available from Hudson Surgical, Inc.

Referring now to FIG. 1A, it should be appreciated that the tool driver 10 of the present invention may be utilized for installing and implanting in bone a prosthesis in any of a number of orthopaedic applications. For example and as shown in FIG. 1A, tool driver 10 may be used to install screws 2 into trauma bone plate 8 of trauma plating assembly 7.

Alternatively, the tool driver 10 may be used to install screw 2A into tibial tray 8A of knee prosthesis 7A.

Alternatively, the tool driver 10 may be used to install screw 2B into plate 8B to form hip screw assembly 7B.

The tool driver 10 may alternatively be used to install screw 2C into acetabular shell 8C of hip cup 7C.

Alternatively, the tool driver 10 may be utilized to install screw 2D into glenoid component 8D of shoulder prosthesis 7D.

Yet another alternative used of the tool driver 10 is to install screw 2E into intermedullary nail 8E to form intermedullary nail assembly 7E.

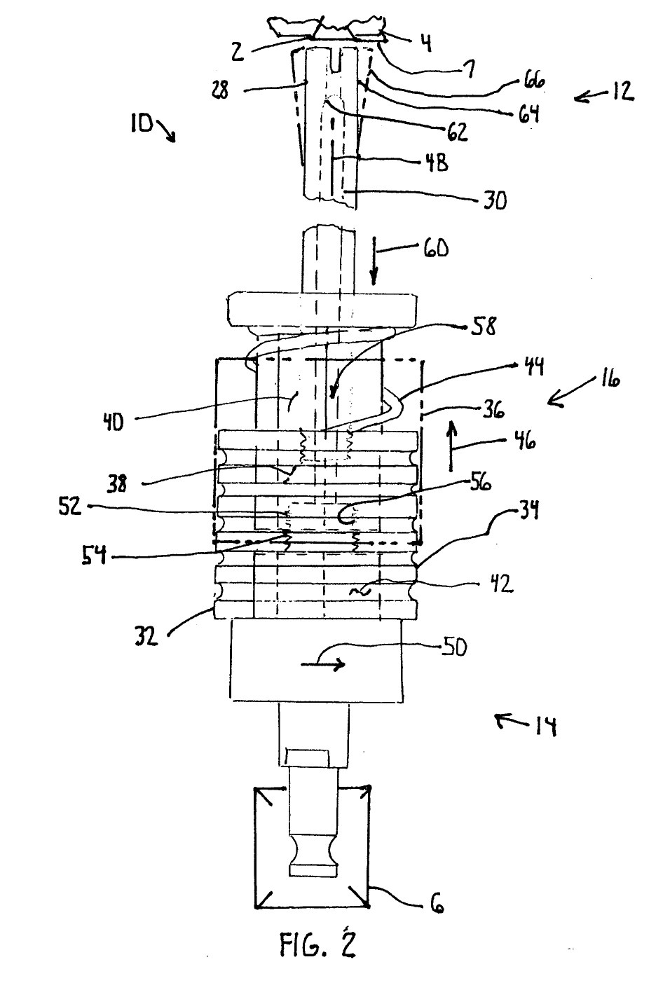

Referring now to FIG. 2, the tool driver 10 is shown in greater detail. The tool driver 10 includes the expandable connector 12, which is used to secure the implant or screw 2 to install the screw 2 into the bone 4. The tool driver 10 further includes the drive connector 14 for connection to power source 6. The tool driver further includes the actuator 16 to selectively and operatively connect the expandable connector 12 to the drive connector 14.

As shown in FIG. 2, the expandable connector 12 may include a flexible member 28. The flexible member 28 is utilized for cooperating with the screw 2. The expandable connector 12 further includes a flexor 30 positioned at least partially within the flexible member 28 for flexing the flexible member 28 into either engagement with the screw 2 or positioned spaced apart from the flexible member 28 to permit disengagement of member 28 from the screw 2.

As shown in FIG. 2, the actuator 16 may include a member 32 in the form of, for example, a collar. The member 32 is operably associated with the expandable connector 12 and with the drive connector 14. The member 32 may have a first relationship 34, as shown in solid, with the expandable connector 12 and the drive connector 14 in which the expandable connector 12 and the drive connector 14 are rotatably connected to each other. The member 32 further has a second relationship 36 with the expandable connector 12 and the drive connector 14 in which the expandable connector 12 and the drive connector 14 are rotatably disconnected from each other.

Referring again to FIG. 2, the member 32 may be in the form of a collar. The collar 32 may define internal flat 38 formed on the collar 32. The driver 32 may also be such that the expandable connector 12 defines external flats 40 formed on the expandable connector 12. Similarly, the drive connector 14 may include external flats 42 formed on the drive connector 14.

The tool driver 10 may be configured to provide for the first relationship 34 in which the expandable connector 12 and the drive connector 14 are rotatably connected and the second relationship 36 in which the expandable connector 12 and the drive connector 14 are rotatably disconnected in any suitable way. For example, as is shown in FIG. 2, the actuator 16 may include a spring 44 which may be, for example connected to the expandable connector 12 and to the collar 32.

To operate the tool driver 10, as shown in FIG. 2, the tool driver operator may advance the collar 32 in the direction of arrow 46 along centerline 48 until the collar 32 moves from first relationship 34 (as shown in solid) to second relationship 36 as shown in phantom. When the collar 32 is in the second relationship 36 as shown in phantom, the collar 32 is rotatably separated from the drive connector 14.

With the collar 32 in the second relationship 36, the operator may rotate the drive connection 14 in the direction of arrow 50 with one hand while resisting with the collar 32 with the other hand. The drive connector 14 includes a stem 52 having external threads 54 which mate with internal threads 56 formed in longitudinal opening 58 of the actuator 16.

As the drive connector 14 rotates in the direction of arrow 50, the actuator 16 is caused to advance in the direction of arrow 60. Then the flexible member 28 is caused to be expanded by the flexor 30. Thus, as the drive connector 14 is rotated in the direction of arrow 50, the flexible member 28 is expanded from its first position 64 as shown in solid to its second position 66 as shown in phantom.

It should be appreciated that if the drive connector 14 is rotated in the direction of arrow 50, until the flexible member 28 is expanded into full engagement with the screw 2, the flexible member 28 will begin to rotate in the direction of arrow 50 while engaged with the screw 2. Therefore, the screw 2 will advance in the direction of rotation of arrow 50 permitting the screw 2 to be hand-tightened by the operator.

Referring now to FIG. 3, the expandable connector 12 is shown in greater detail. The expandable connector 12, as is shown in FIG. 3, may include flexible member 28 made of, for example, a resilient material. The flexible member 28 may include a transverse slit 68 located on end 70 of the flexible member 28. Flexible member 28 may have a solitary slit 68 or a plurality of spaced apart slits.

The flexible member 28 may be hollow or be defined by an internal cavity 72. The cavity 72 may be used for receiving the flexor 30. The flexor 30 may include a bullet tapered or pointed shape end or nose 74. The cavity 72 may have a similar bullet-tapered or pointed end 76 for cooperation with the pointed end 74 of the flexor 30.

Flexible member 28 may include an external periphery 78, which expands to connect with, for example, internal socket head 3 of the screw. The head 6 may have any suitable shape, such as a star shape, a Torx® shape, or a polygon shape, for example a square internal head or a hexagonal head. It should be appreciated that the external periphery 78 of the flexible member 28 preferably has a shape conforming to that of the internal head 3 of the screw 2.

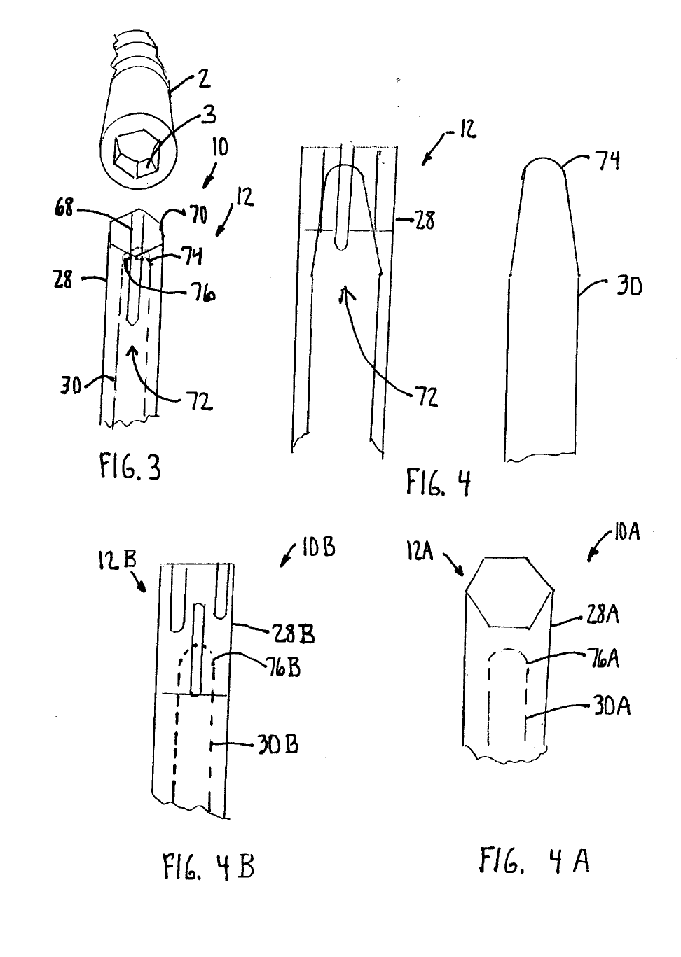

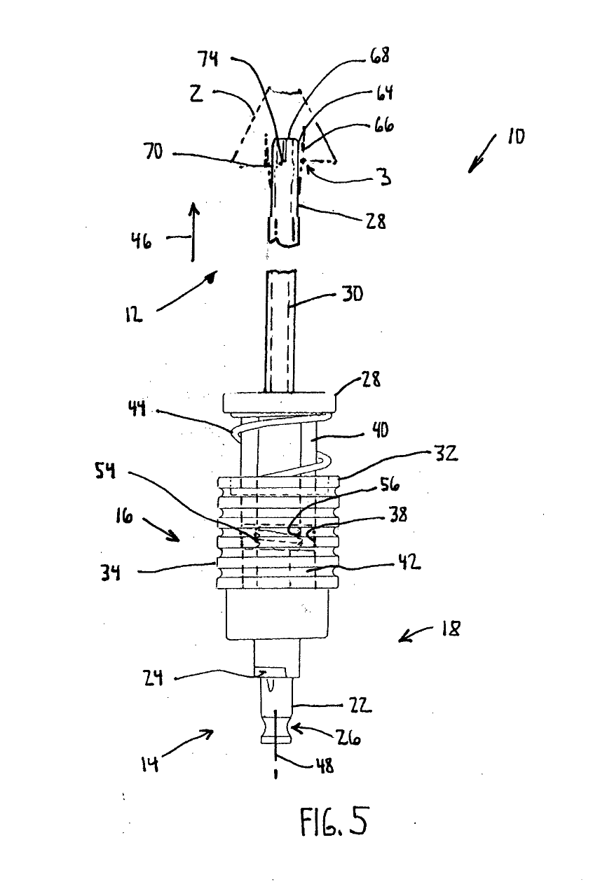

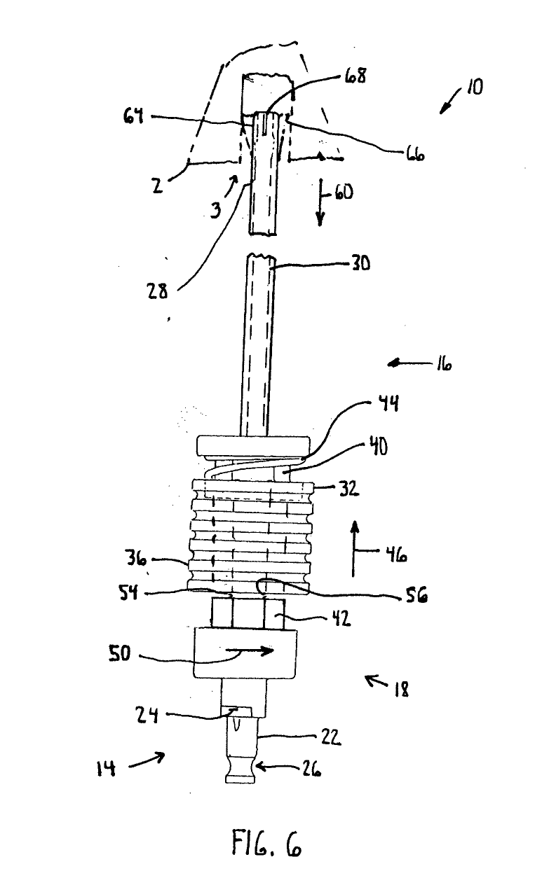

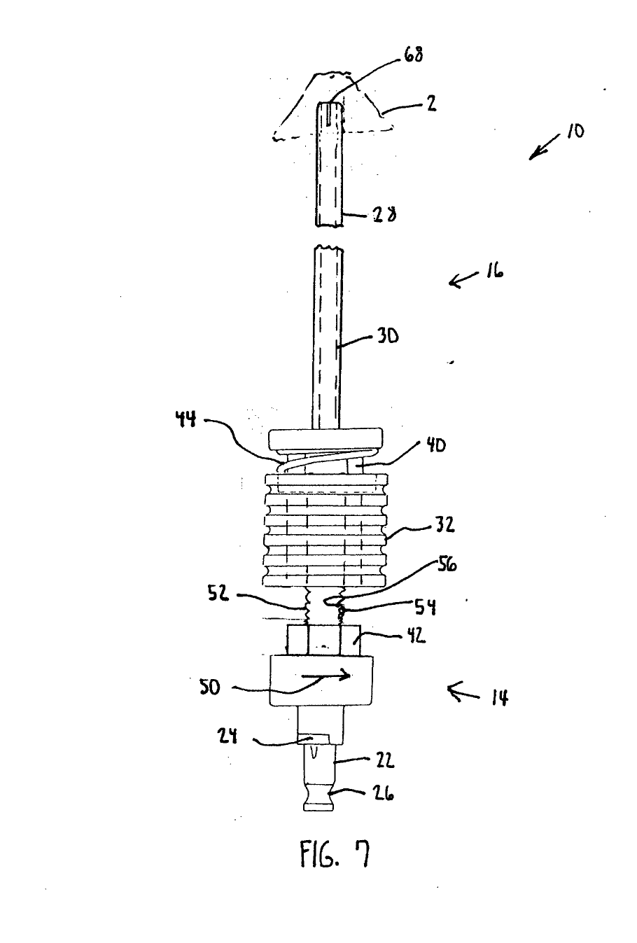

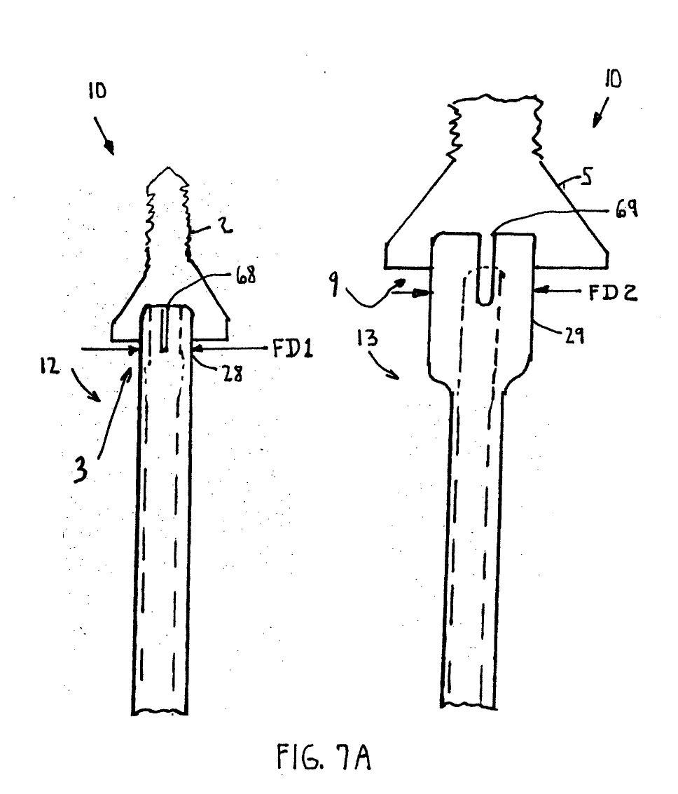

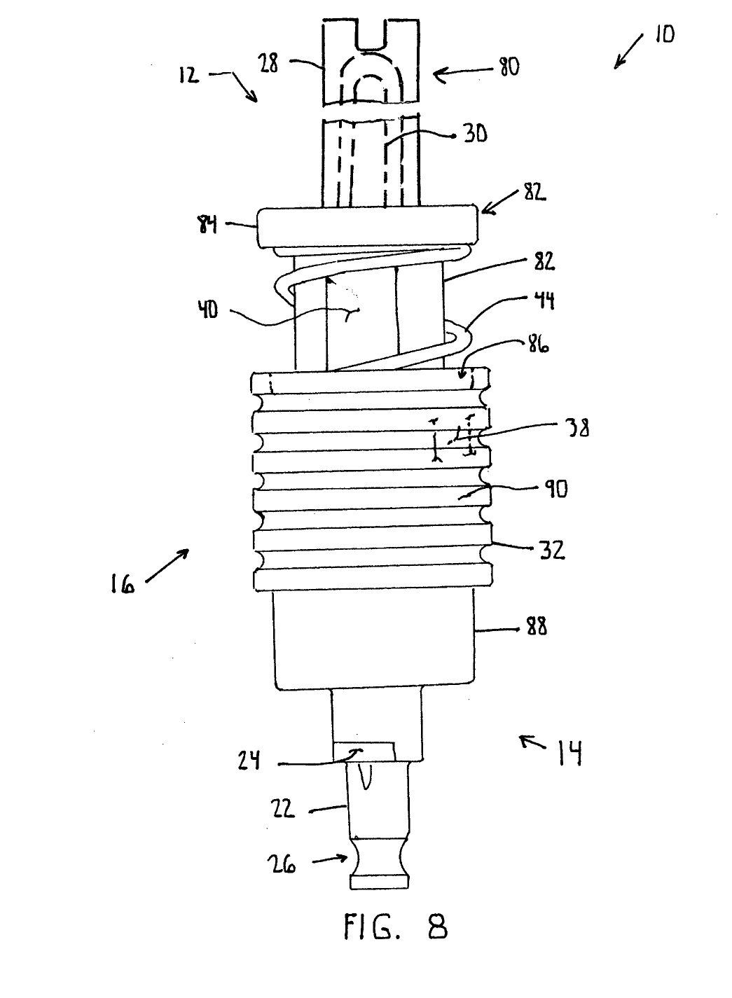

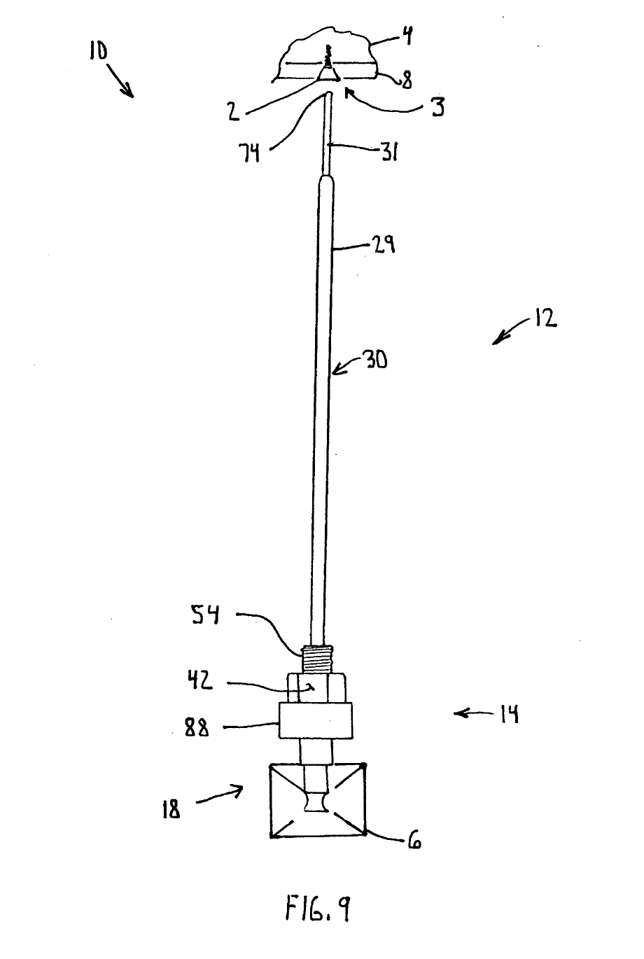

Referring now to FIG. 4, the flexible member 28 and the flexor 30 are shown in a disengaged or separate view as separate components.

Referring now to FIG. 4A, another embodiment of the present invention is shown as tool driver 10A. The tool driver 10A includes a flexible member 28A in the form of for example, a bladder. The bladder 28A is expanded by, for example, a flexor 30A, which fits within the cavity 76A formed in the flexible member 28A.

Referring now to FIG. 4B, yet another embodiment of the present is shown as tool driver 10B. The tool driver 10B includes an expandable connector 12B, which includes a flexible member 28B in the form of a collet. The collet 28B is expanded by flexor 30B, which fits within cavity 76B. The collet 28B is expanded by the flexor 30 fitted within the cavity 76B of the flexible member 28.

Referring now to FIGS. 5-7, tool driver 10 is shown in FIGS. 5, 6 and 7 in first, second, and third operating modes, respectively.

Referring now to FIG. 5, tool driver 10 is shown in the installation position in which the tool driver 10 is advanced in the direction of arrow 46 along longitudinal centerline 48 until flexible member 28 of the expandable connector 12 is fitted into internal socket 3 of the implant or screw 2.

In the configuration of the tool holder 12 of FIG. 5, the external periphery 70 of the flexible member 28 is in clearance with the internal socket 3 of the implant screw 2 so that the tool driver 10 may be installed into position with the screw 2. In this position, the collar 32 is in first relationship 34 such that the drive connector 14, the actuator 16 and the expandable connector 12 are all fixedly secured to each other. In this relationship 34, the internal flats 38 of the collar 32 are engagement with external flats 42 of the drive connector 14 and with the external flats 40 of the expandable connector 12.

Referring now to FIG. 6, the tool driver 10 is shown in the second operating mode in which flexible member 28 of the tool driver 10 is fixedly secured to the internal socket 3 of the screw 2.

In the configuration as shown in FIG. 6, the operator may manually tighten the flexible member 28 of the tool driver 10 to the internal socket 3 of the screw 2. As shown in FIG. 6, the collar 32 of the actuator 16 is in its second relationship 36 in which the operator advances the collar 32 in the direction of arrow 46 such that spring 44 is compressed and the collar 32 advances such that the internal flats 38 and the collar 32 are separated from external flats 42 of the drive connector 14 such that the drive connector 14 may be rotated in the direction of arrow 50 with respect to the collar 32.

As the drive connector 14 is rotated in direction of arrow 50, the internal thread 56 of the flexible member 28 of the expandable connector 12 cooperates with extended threads 54 of drive connector 14 to advance the flexible member 28 in the direction of arrow 60. As the flexible member 28 moves in the direction of arrow 60, the flexible member 28 cooperates with the flexor 30 to expand the flexible member 28 from its first position 64 as shown in solid to its second position 66 as shown in phantom. Once the flexible member 28 expands to the second position 66, the flexible member 28 is securely supporting the implant or screw 2.

It should be appreciated that if the drive connector 14 continues to be rotated in the direction of arrow 50, once the screw 2 is fully engaged with the flexible member 28 the flexible member 28 rotates with the flexor 30 as well as with the drive connector 14 to cause the screw 2 to rotate in the direction of arrow 50. Thus, in the second condition as shown in FIG. 6, not only can the flexible member 28 of the tool driver 10 be actuated to tighten the flexible member 28 of the tool driver 10 to the screw 2, the tool driver 10 in the condition as shown in FIG. 6 may be used to tighten the screw 2 to bone 4.

Referring now to FIG. 7, the tool driver 10 is shown in the third operating condition. As shown in FIG. 7, when the tool driver is in the third operating position, the actuator 16 and the collar 32 are in first relationship 34. In the first relationship 34, the collar 32 is positioned in the condition with the spring 44 urging the collar 32 against the drive connector 14. In the first relationship 34 the internal flats 38 of the collar 32 are in engagement with the external flats 40 of the flexible member 28 of the expandable connector 12 and are in engagement with the external flats 42 of the drive connector 14.

In the first relationship 34, the drive connector 14, actuator 16 and the expandable connector 12 are fixedly and rigidly connected. In the third condition as shown in FIG. 7, the power source 6 may apply torque to the tool driver 10, and thus, to the screw 2 to install it in its final position with respect to the bone 4.

Referring now to FIG. 7A, the tool driver 10 may further include a third component in the form of, for example, second expandable connector 13. Connector 13 may have any suitable configuration and may, as shown in FIG. 7A, be in the form of a flexible member 29 that mates with flexor 30. The flexible member 29 is similar to the flexible member 28 except that the flexible member 28 has a flexible member diameter FD-2, which is different than the flexible member diameter FD-1 of the first mentioned flexible member 28 of the expandable connector 12.

The flexible member 29 preferably defines an internal cavity 77 having a shape similar to the cavity 76 of the flexible member 28. By having the cavity 77 be similar to cavity 76, the flexor 30 may be used with both the first mentioned flexible member 28 and the second flexible member 29. The tool driver 10 may then be a tool driver that can accommodate the first screw 2 having a first internal socket 3 with a second implant or screw 5 having a second and different internal socket 9 of different dimensions.

Thus, for the tool driver 10 to be converted from one for tightening a first screw 2 to one for tightening the second screw 5, the tool driver 10 merely needs to have the first mentioned flexible member 28 of the expandable connector 12 of the tool driver 10 replaced with second flexible member 29.

It should be appreciated that the flexible member 29 of the tool driver 10 may, like the flexible member 28, include a slit, for example slit 69.

Referring now to FIG. 8, the tool driver 10 is shown in greater detail. The tool driver 10 includes the drive connector 14, the actuator 16, and the expandable connector 12.

The expandable connector 12 includes the flexible member 28 and the flexor 30, which is used to flex or expand the flexible member 28. The flexible member 28, as shown in FIG. 8, includes a tube portion 80, which includes the expandable portion of the flexible member 28 as well as a spool portion 82 connected to the tube portion 80.

The spool portion 82 includes the external flats 40 which mate with the internal flats 38 of the collar 32. The spool portion 82 includes a flange 84, which with recess 86 formed in the collar 32 serve to contain the spring 44, which urges the collar 32 in contact with body 88 of the drive connector 14. The collar 32 may include a periphery 90, which includes features for assisting in grasping the collar 32.

Referring now to FIG. 9, the drive connector 14 and the flexor 30 of the tool driver 10 are shown connected to each other. It should be appreciated that the drive connector 14 and the flexor 30 may be integral or may be fixedly secured to each other. The flexor 30 forms a portion of expandable connector 12.

The flexor 30 includes a base portion 27 and a tip portion 31 extending from the base portion 29. The tip portion 31 defines tip or end 74 for cooperation with the flexible member 28 of the expandable connector 12. The drive connector 14 includes the body 88 as well as flats 42. The drive connector 14 also includes external threads 54 as well as a drive adapter 18 for cooperation with power source 6.

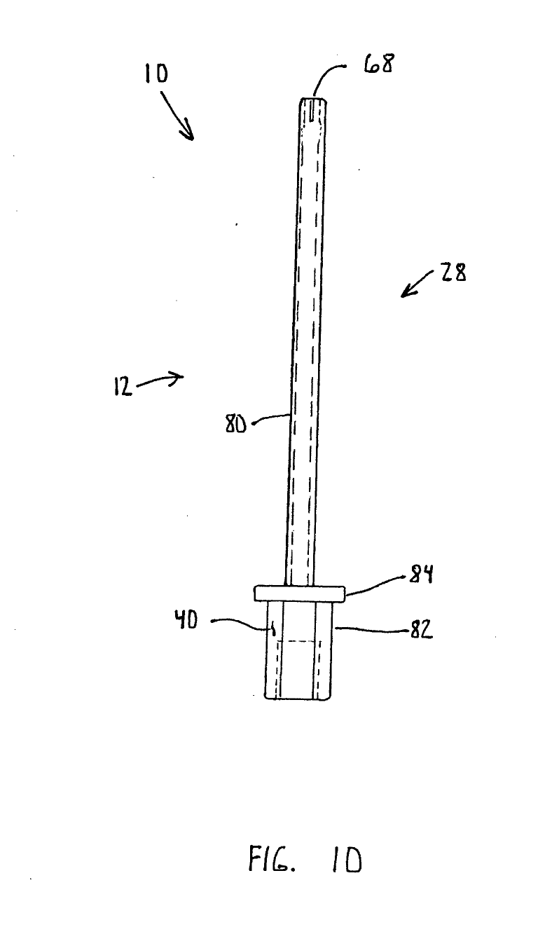

Referring now to FIG. 10, the flexible member 28 of the expandable connector 12 of the tool driver 10 is shown in greater detail. The flexible member 28 includes the hollow tube portion 80 as well as the spool portion 82. It should be appreciated that the hollow tube portion 80 and the spool portion 82 may be integral with each other. Alternatively, the hollow tube portion 80 may be fixedly secured to the spool portion 82.

The spool portion 82 as shown in FIG. 10 includes flange 84 as well as external flats 40. The hollow tube portion 80 as shown in FIG. 10 includes slits 68 to provide for the expansion of the hollow tube portion 80 of the flexible member 28.

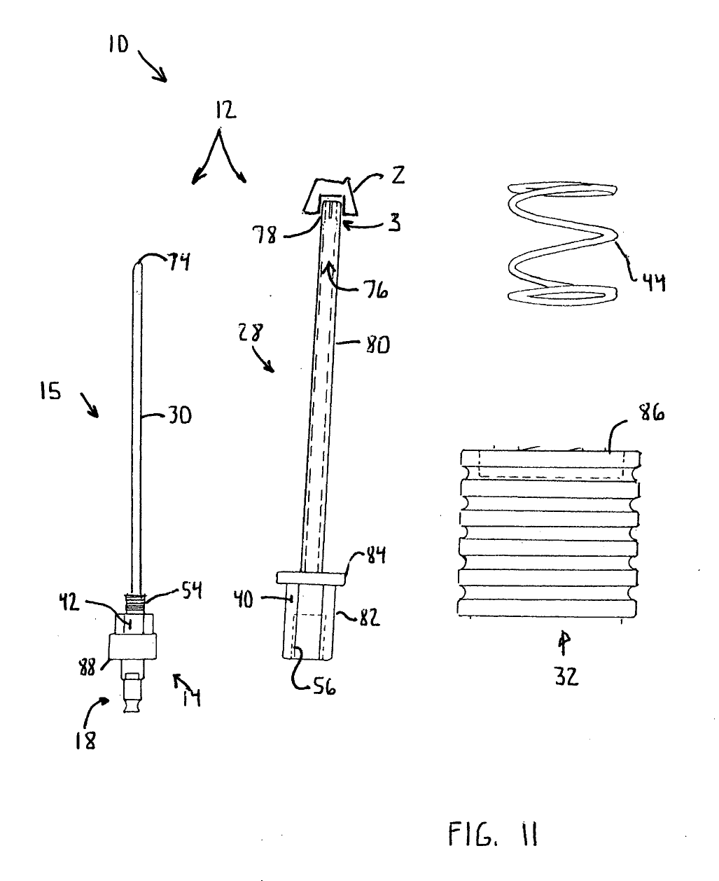

Referring now to FIG. 11, the tool driver 10 may according to the present invention be assembled from components that may be easily assembled and disassembled, cleaned, and sterilized. For example and as shown in FIG. 11, a tool driver 10 may include separate components including a connector flexor component 15, a flexible member 28, a collar 32 and a spring 44.

Each of the four components, the connector flexor component 15, the flexible member 28, the collar 32 and the spring 44, may be made of any suitable or durable material that is sterilizable by any commercially available sterilizing procedure. The tool driver 10, including the component 15, member 28, collar 32 and spring 44, may be made of any suitable durable material for example a ceramic, a plastic, a composite, or a metal. If made of a metal, the components of the tool driver 10 may be made of, for example, a cobalt chromium alloy, a stainless steel alloy, or a titanium alloy.

The connector flexor component 15, as is shown in FIG. 11, includes the drive connector 14 and the flexor 30, which extends from the drive connector 14. The drive connector 14 includes the body 88 and the drive adapter 18 extending from the body 88. The drive connector 14 further includes the external flats 42, which extend from the body 88 in a direction opposed to the drive adapter 18. The driver connector 14 further includes the external threads 54. The flexor 30 defines an end 74 for cooperation with the expandable connector 12.

The flexible member 28 includes the spool portion 82 and the hollow tubular portion 80, which extends from the spool portion 82. The spool portion 82 includes the flange 84 as well as external flats 40 and internal threads 56. The tubular portion 80 defines an internal cavity 76 thereof as well as an external periphery 78 for cooperation with the internal flats 3 formed on implant or screw 2.

While the tool driver 10 of the present invention may be designed, as is shown in FIG. 11, with the drive connector and flexor combined into a common component and with the spool portion 82 and the tube portion 80 connected to form the flexible member 28, it should be appreciated that these components may in fact be separable components which may be fitted to each other to form the tool driver of the present invention.

For example and as shown in FIG. 11A, the tool driver of the present invention may be in the form of tool driver 10A. The tool driver 10A may include a collar and spring (not shown) identical to the collar 32 and the spring 44 of the tool driver 10 of the present invention.

The tool driver 10A may include a drive connector 14A, which is a separate component from the flexor 30A. The drive connector 14A and the flexor 30A may be slidably connected to each other to form a subassembly 15A. The component 30A may have generally the same dimensions as the flexor 30A of the tool driver 10 of FIG. 11. Similarly, the drive connector 14A may have dimensions substantially the same as the drive connector 14 of the tool driver 10 of FIG. 11.

Similarly, the tube 80A of the tool driver 10A of FIG. 11A may, as shown in FIG. 11A, be a separate component from the spool 82A of the tool driver 10A of FIG. 11A. The tube 80A and the spool 82A may be slidably connected to each other. The tube 80A may have dimensions substantially similar to the tube portion 80 of the flexible member 28 of FIG. 11. The spool 82A of the tool driver 10A of FIG. 11A may have a size and shape substantially the same as the spool portion 82 of the flexible member 28 of the tool driver 10 of FIG. 11.

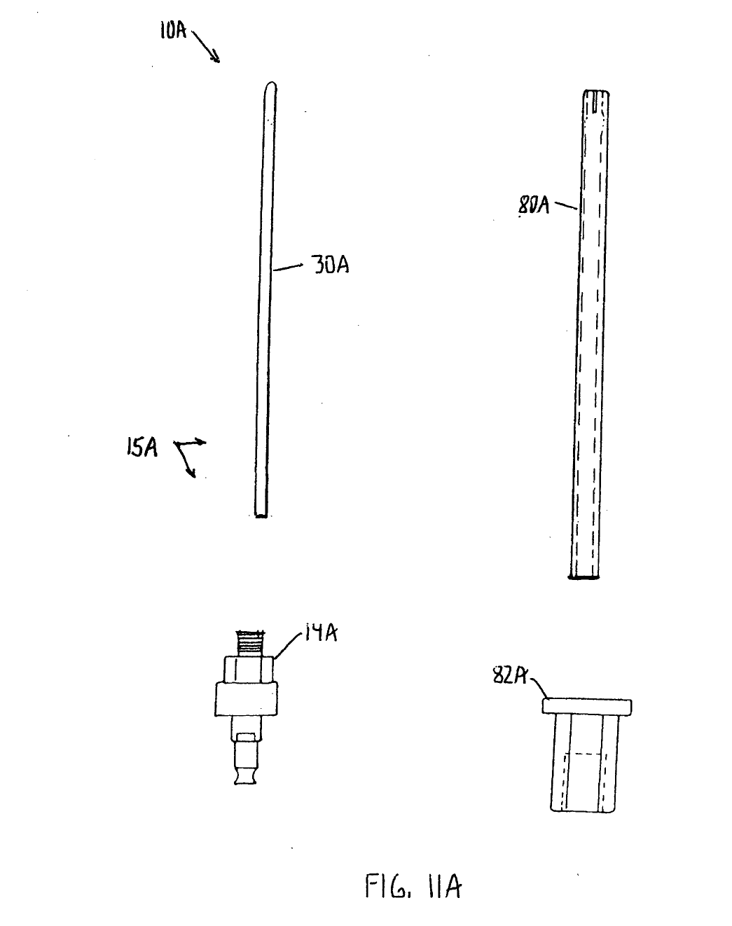

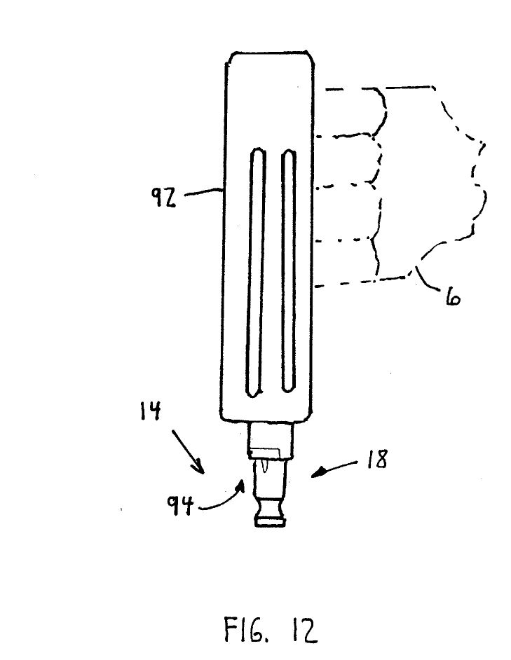

According to the present invention and referring now to FIG. 12, the tool driver 10 may further include a handle 92 for manually operating the tool driver 10 by, for example a surgeon or other medical professional. The handle 92 includes a handle adapter 94 in the form of for example a cavity for receiving the drive adapter 18 of the drive connector 14 of the tool driver 10.

Referring now to FIG. 13, the spring 44 is shown in greater detail. The spring 44 may be constructed as a helical wire having a wire diameter WD. The spring 44 may be further defined by a spring diameter DS as well as a free-length FL of the spring 44.

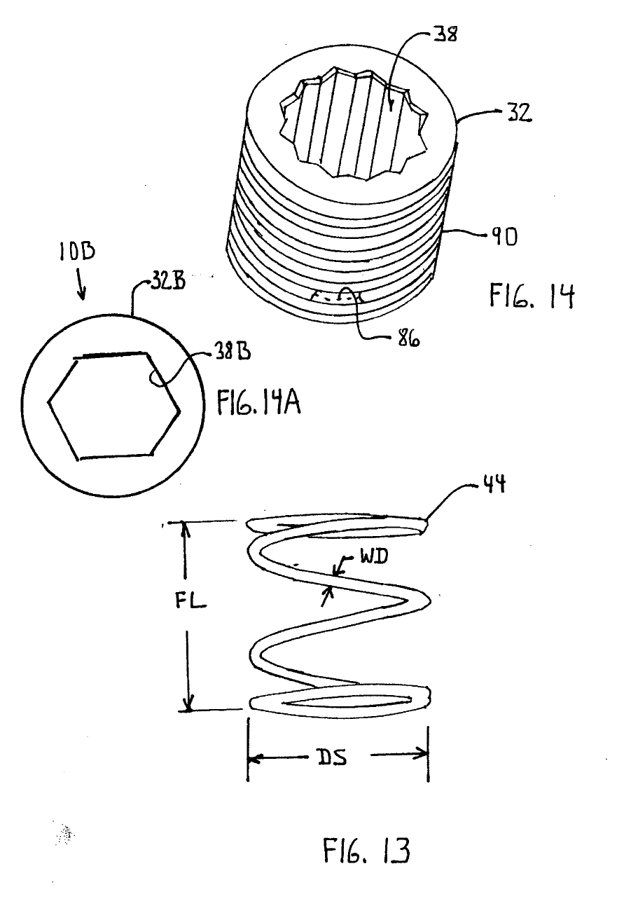

Referring now to FIG. 14, the collar 32 is shown in greater detail. The collar 32 includes a plurality of internal flats 38 as well as a recess 86 for receiving the spring 44. The collar 32 further defines periphery 90 thereof. The periphery 90 may include features on the periphery for assisting in holding the collar 32. The flats 38 may, as shown in FIG. 14, include a large number of flats, for example 32 flats.

Referring now to FIG. 14A, an alternate embodiment of the present invention is in the form of tool driver 10B. The tool driver 10B includes a collar 32B that is different than the collar 32 of the tool driver 10 of FIG. 8 in that the collar 32 includes a set of internal flats 38B, which are different than the internal flats 38 of the collar 32 of FIG. 14. The internal flats 38B of the collar 32 form a hexagonal shape.

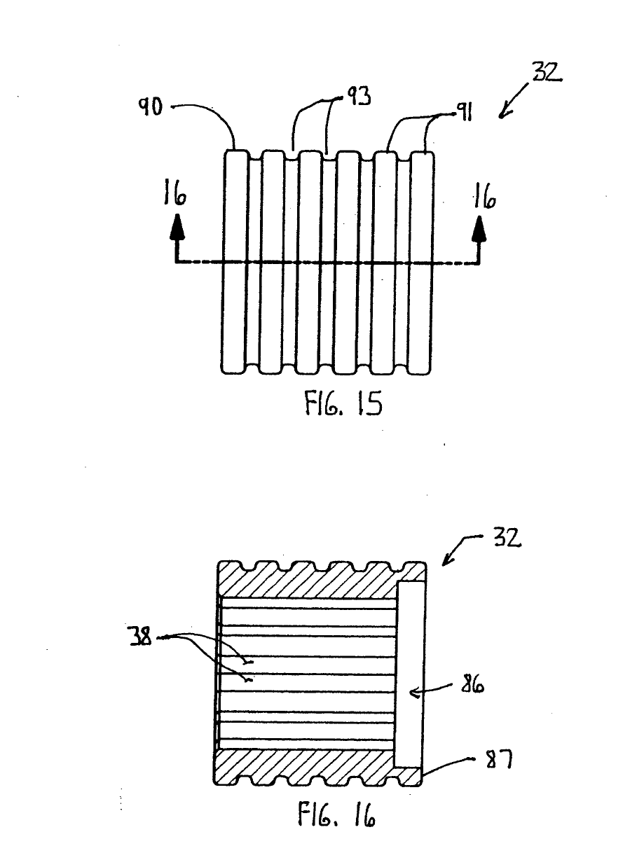

Referring now to FIGS. 15, 16 and 17, the collar 32 is shown in greater detail. The collar 32 as shown in FIG. 15 includes a periphery 90, which is in the form of cylindrical rings 91 with grooves 93 formed there between. It should be appreciated that the periphery 90 may be in the form of additional configurations in the form of for example knurls or splines or may include an abrasive coating to assist in the grasping of the collar 32.

The collar 32 as shown in FIG. 16 includes recess 86 formed on an end 87 of the collar 32. The recess 86 is utilized to contain an end of the spring 44.

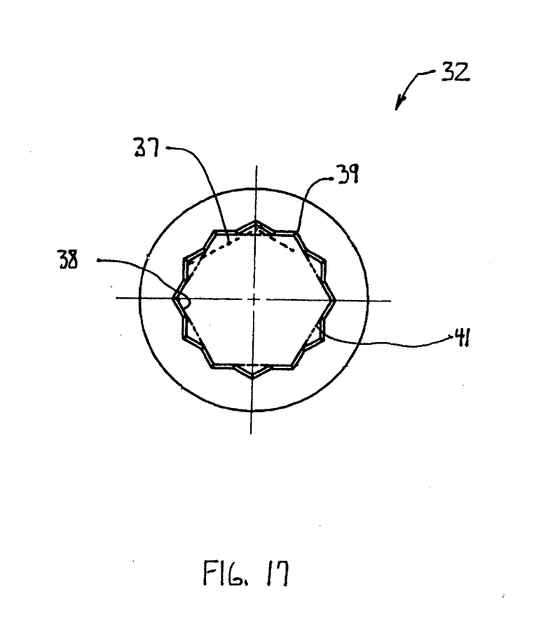

Referring now to FIG. 17 the flats 38 of the collar 32 are shown in greater detail. The flat 38, as shown in FIG. 17, are in the form of 24 flats. The flats 38 form adjacent pairs 39 of flats 38, which define angle β there between. Every other adjacent pair 39 of the flats 38 is utilized to receive a hexagonal external edge.

For example and as shown in FIG. 17, the flats 38 are adapted for receiving a first hexagonal shape 37 as shown with a dashed line and a second hexagonal shape as defined by phantom line 41. The flats 38 of FIG. 17 assist in minimizing the rotation required for the collar 32 to engage in a particular set of flats on the mating parts.

As can be seen in FIG. 17, the dash-line 37 is in contact with twelve of the twenty-four flats 38 and the phantom shape 41 is in contact with the other twelve of the twenty-four flats 38.

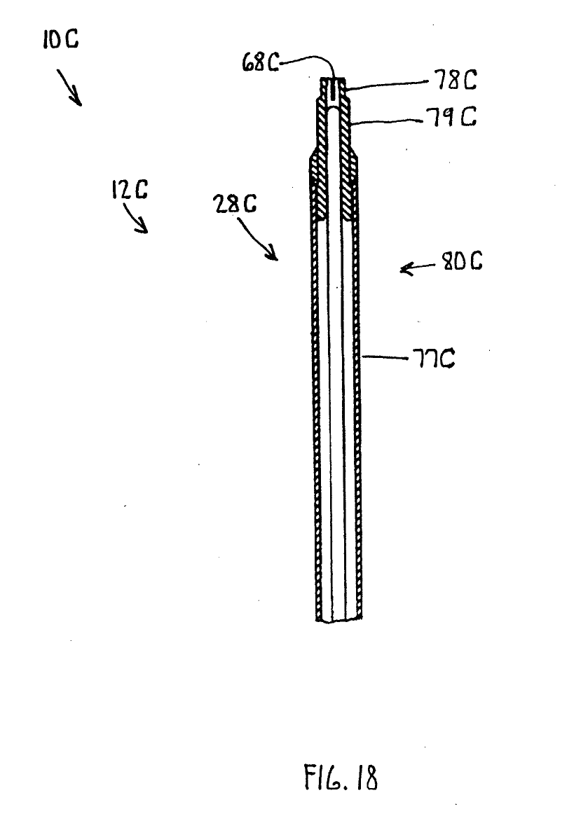

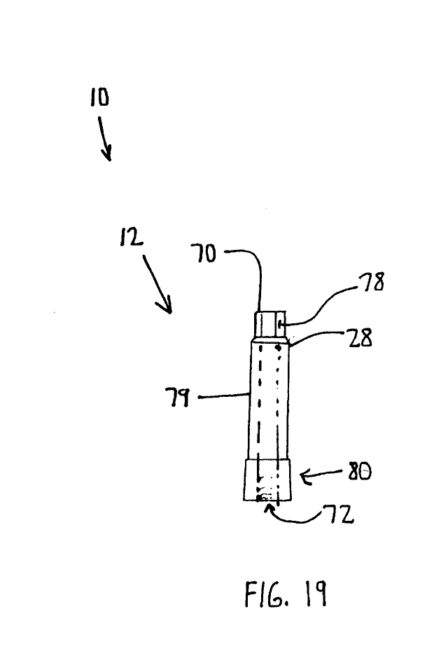

Referring now to FIG. 18, another embodiment of the tool driver of the present invention is shown as tool driver 10C. Tool driver 10C is similar to tool driver 10 of FIG. 8 except that tool driver 10C includes an expandable connector 12C which includes a flexible member 28C that is somewhat different than the flexible member 28 of the tool driver 10 of FIG. 8 in that the flexible member 28C includes a hollow tube portion 80C that is modular or has a two-piece construction. The hollow tube 80C includes a face 77C and a separate removable tip 79C, which is removable from the base 77C. The tip 79C includes a surface 78C for cooperating with the screw 2. The tip 79C can easily be replaced with a different tip that has a different cooperating surface to accommodate a larger or smaller screw.

Referring now to FIG. 19, tip portion 79 of the flexible member 28 of the expandable connector 12 of the tool driver 10 is shown in greater detail. The tube portion 80 includes an external periphery 78 with adjacent end 70 to cooperate with the screw 2. The tube portion 80 includes an internal cavity 72 for receiving the flexor 30.

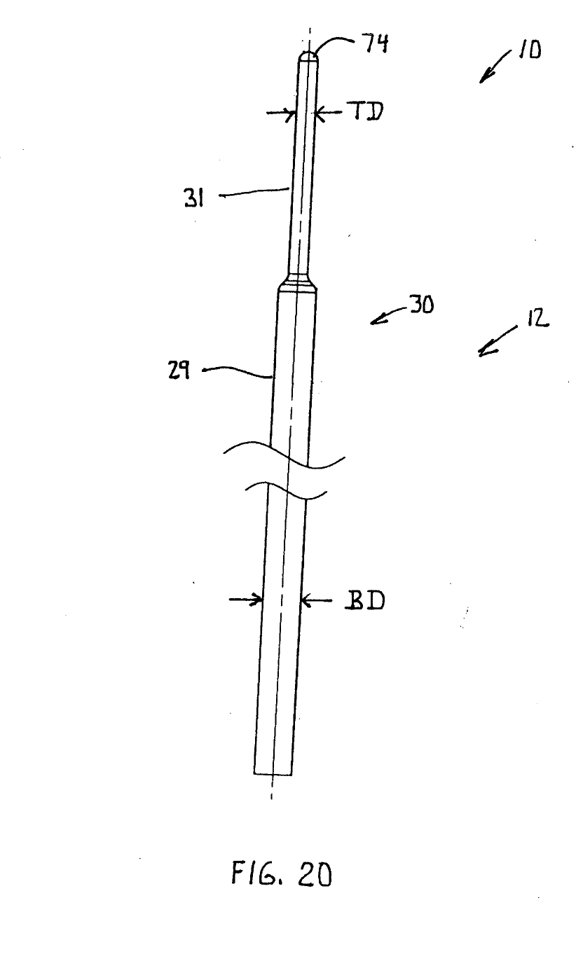

Referring now to FIG. 20, the flexor 30 of the expandable connector 12 of the tool driver 10 is shown in greater detail. The flexor 30 may, as shown in FIG. 20, be connected to the drive connector 14. The flexor 30 as shown in FIG. 20 includes a base portion 27 secured to the drive connector 14 as well as a tip portion 31 connected to the base portion 27. The tip portion 31 includes the point or end 74 for cooperation with the hollow-tube portion 80 of the flexible member 28. The base portion 27 is defined by a base diameter BD while the tip portion 31 is defined by a tip diameter TD, which as shown in FIG. 20 may be smaller than the base diameter BD.

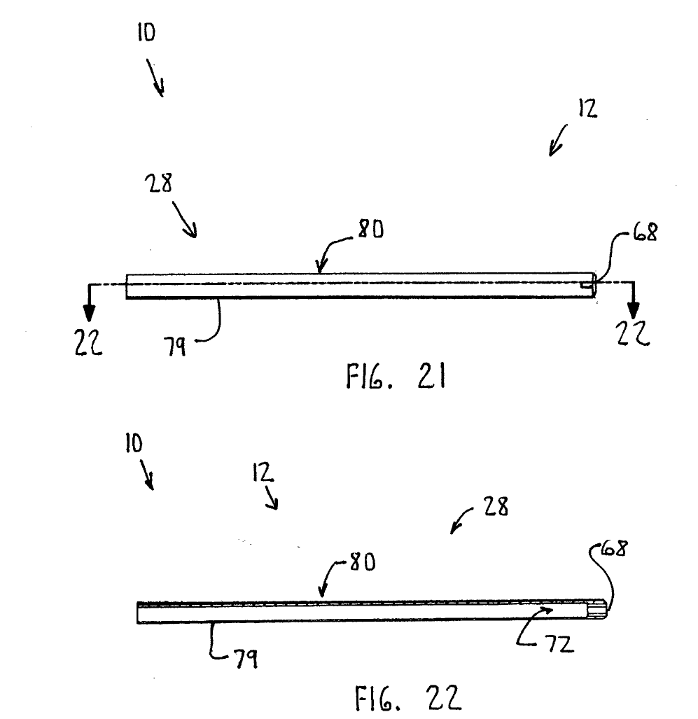

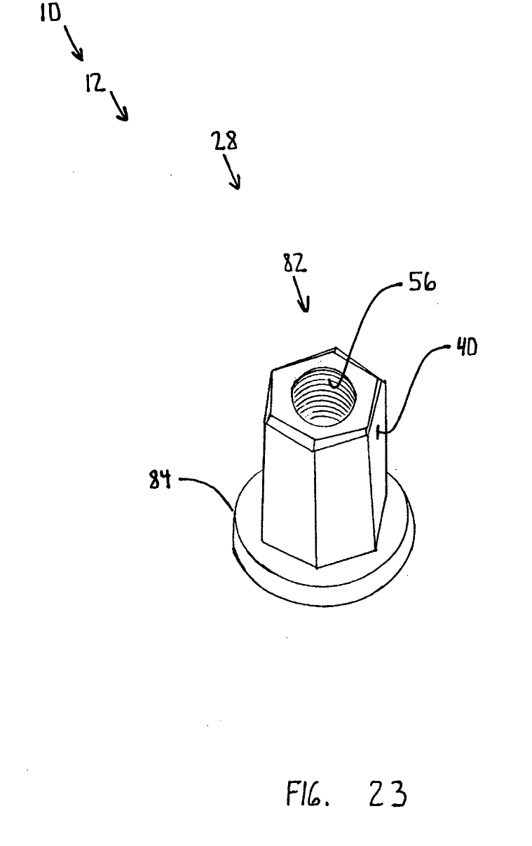

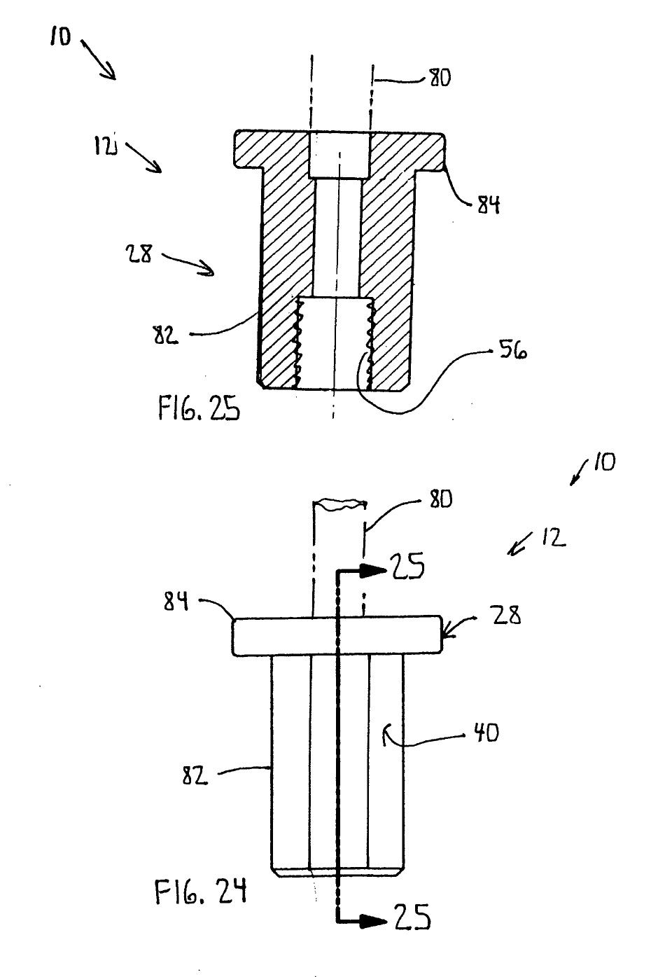

Referring now to FIGS. 21 and 22, the tip portion 79 of the hollow tube portion 80 of the flexible member 28 of the expandable connector 12 of the driver 10 is shown in greater detail. The tip portion 79 includes a central cavity 72 for receiving the flexor 30 of FIG. 20. The tip portion 79 further includes a slit 68 for permitting periphery 78 of the tip portion 79 to expand when contacted with flexor of FIG. 20.

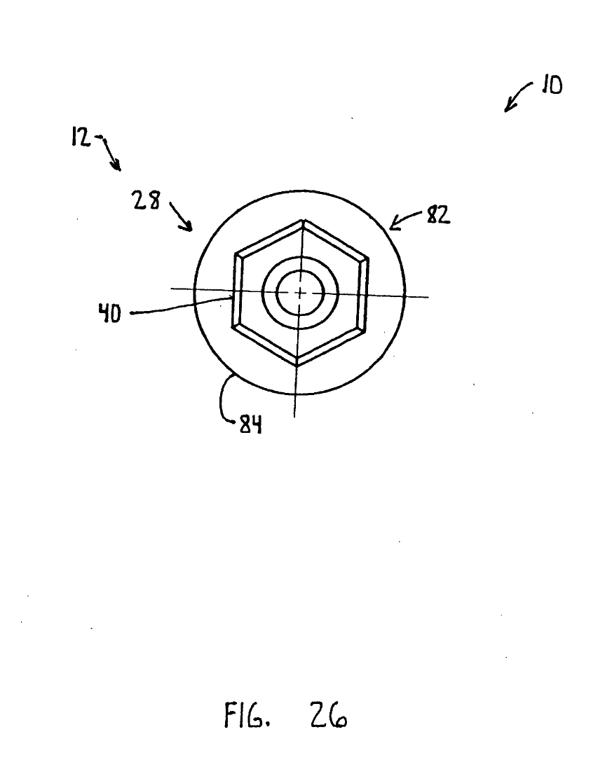

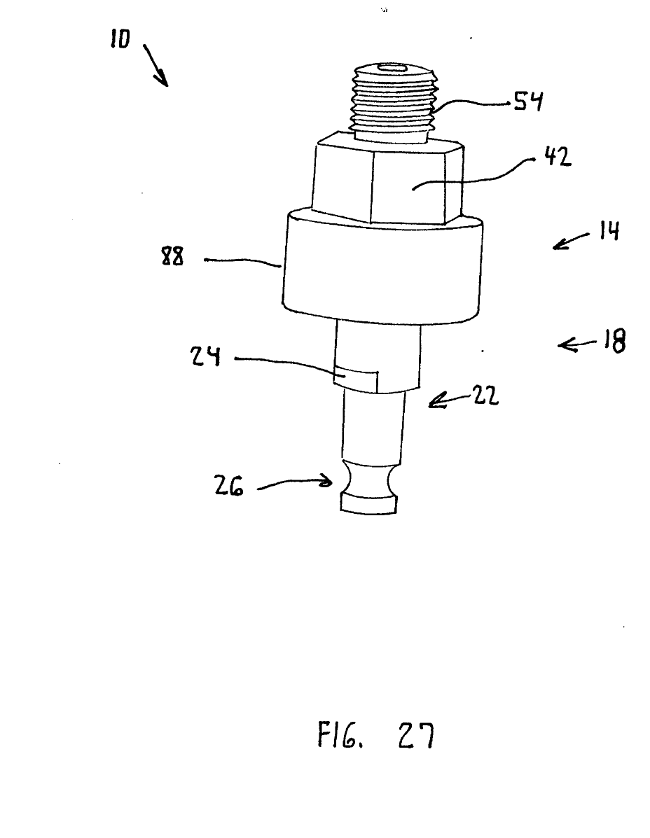

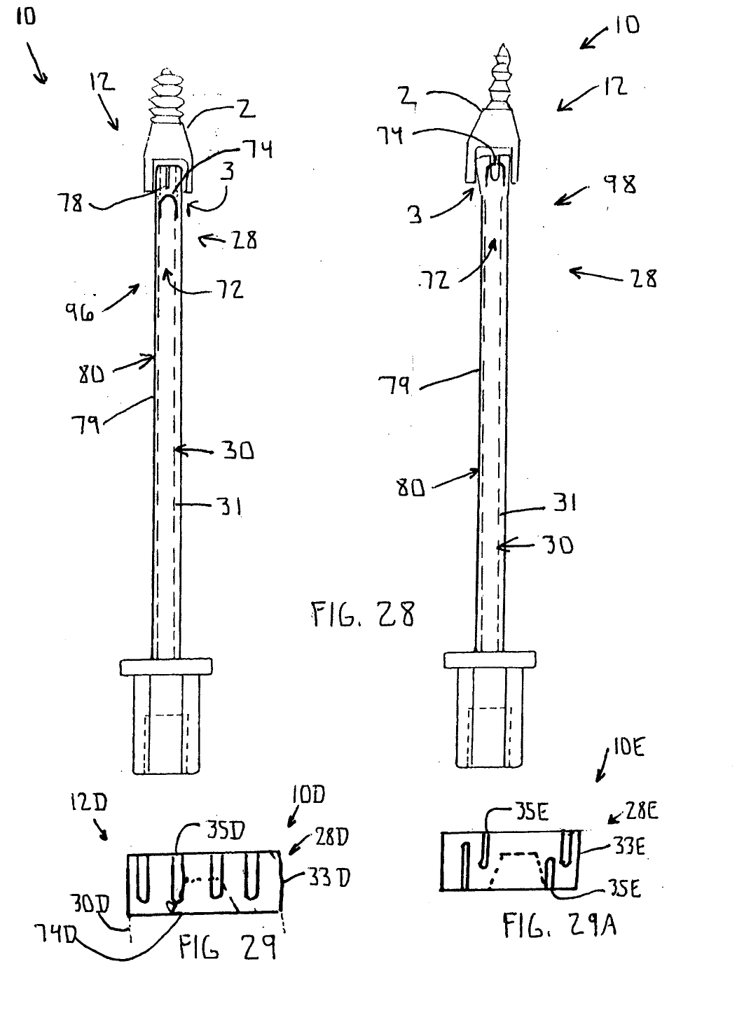

Referring now to FIGS. 23-26, the spool portion 82 of the flexible member 28 of the expandable connector 12 of the tool driver 10 is shown in greater detail. The spool portion 82 as shown in FIG. 23-26 is connected to tube portion 80 of the flexible member 28. The spool portion 82 and the tube portion 80, it should be appreciated, may alternatively be integral with each other.

The spool portion 82 may, as is shown in FIG. 23-26, include a flange 84 for constraining the spring 44 of the tool driver 10 as well as internal threads 56 for cooperation with external threads 54 of the drive connector 14. The spool portion 82 may further include a flat or, as shown in FIG. 23-26, a plurality of flats 40. The flats 40 shown in FIG. 26 may form a hexagonal periphery. The flats 40 cooperate with the internal flats 38 of the collar 32 of FIG. 8.

Referring now to FIG. 27, the drive connector 14 of the tool driver 10 is shown in greater detail. The drive connector 14 includes the body 88 from which the drive adapter 18 extends. The drive adaptor 18 includes a stem 22 defining a circumferential groove 26. The drive adapter 18 also includes a flat, for example spaced apart flats 24. The drive connector 14 further includes a portion having, for example, six-sided hexagonal exterior flats 42 positioned opposed to the drive adaptor 18. The drive connector 14 also includes external threads 54 for cooperation with the internal threads 56 of the spool portion 82 of the expandable connector 12 of FIGS. 23-26.

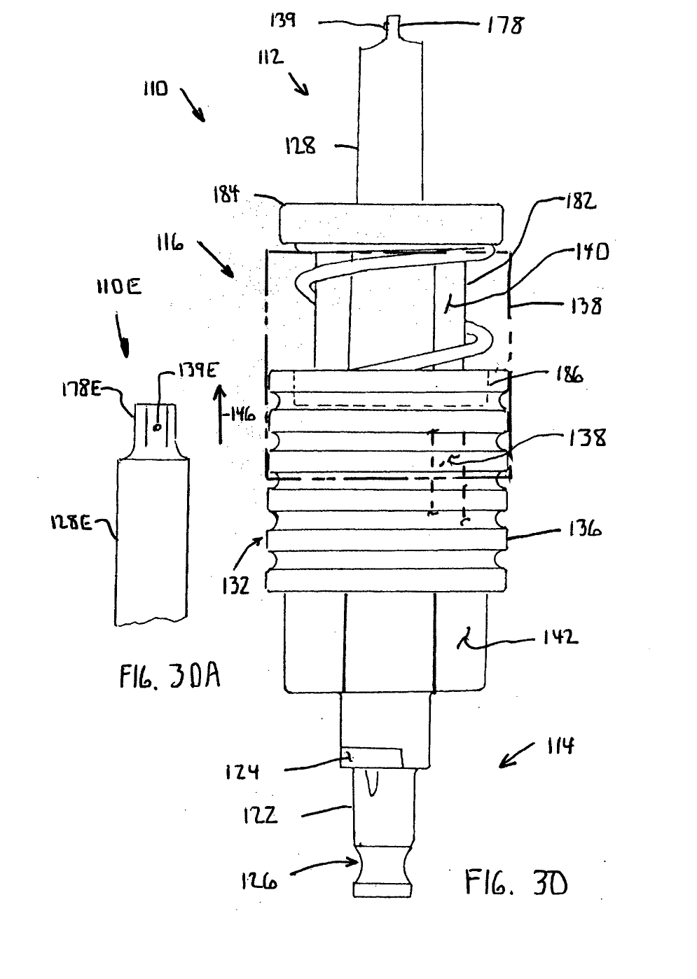

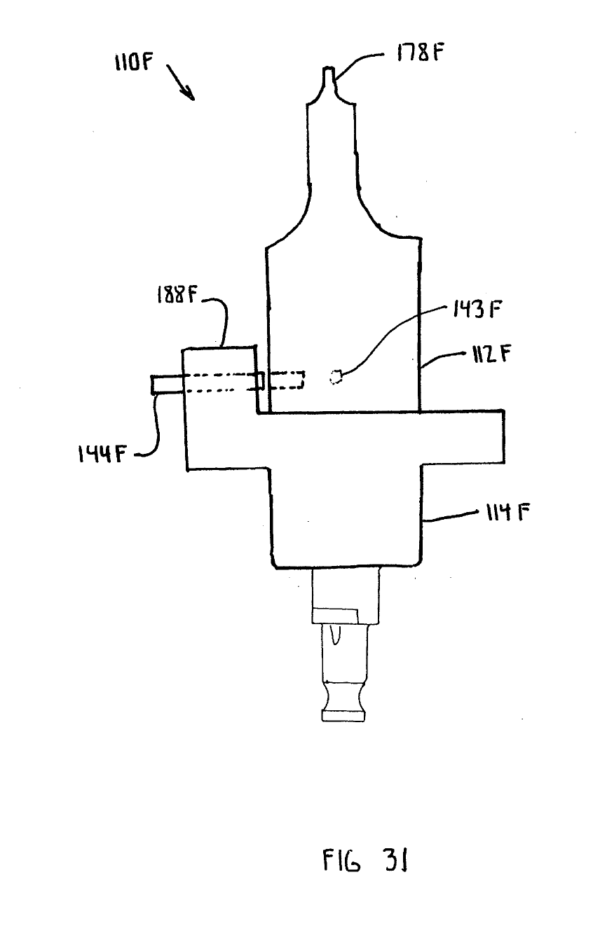

Referring now to FIG. 28, the expandable connector 12 is shown both in its relaxed state 96 and its actuated state 98. Referring now to the relaxed state 96 of the expandable connector 12 of the tool driver 10, periphery 78 of the tip portion 79 of the tube portion 80 of the flexible member 28 is in a spaced apart relationship with the cavity 3 of the screw 2 so that the tool driver 10 may be inserted into the cavity 3 of the screw 2. The tip portion 31 of the flexor 30 is slidably fitted into cavity 72 of the tip portion 79 of the expandable connector 12. The flexible member 28 is spaced from the screw 2 because the point 74 of the tip portion 31 of the flexor 30 is spaced from the expandable connector 12. Thereby the flexor 30 does not expand the expandable connector 12 of the tool driver 10.

Now referring to the activated state 98 of the expandable connector 12 of the tool driver 10, the tip portion 31 of the flexor 30 is shown with the point 74 of the tip portion 31 engaging with the flexible member 28 such that the periphery 78 of the tip portion 79 of the tube portion 80 of the expandable flexible member 28 is expanded into engagement with cavity 3 of the screw 2 such that the flexible member 28 engages screw 2 such that the tool driver 10 may be utilized to install the screw 2 into bone 4.

Referring now to FIG. 29, yet another embodiment of the present invention is shown as tool driver 10D. The tool driver 10D is similar to the tool driver 10 of FIG. 8 except that the tool driver 10D includes an expandable connector 12D having a flexible member 28D, which is different than the flexible member 28 of the tool driver 10. In fact, the flexible member 28D includes collet 33D, which engages with point 74D of the flexor 30D to actuate collet 33D. Collet 33D includes a plurality of cuts or slits 35D which serve to make collet 33D flexible. Collet 28D may be in the form of a collet with a post-slit 35D as shown in FIG. 29.

Referring now to FIG. 29A, yet another embodiment of the present invention is shown as tool driver 10E. The tool driver 10E includes flexible member 28E that has a collet 33E with opposed slots 35E.

Referring now to FIG. 30, yet another embodiment of the present invention is shown as tool driver 110. Tool driver 110 is similar to the tool driver 10 of FIG. 8 except that the tool driver 110 does not include an expandable member.

As shown in FIG. 30, the tool driver 110 includes a drive connector 114 similar to the drive connector 14 of the tool driver 10 of FIG. 8. The drive connector 114 includes a stem 122 having a circumferential groove 126. The drive connector 114 further includes spaced-apart parallel flats 124. The drive connector 114 further includes external flats 142 that cooperate with internal flats 138 formed in collar 132. Collar 132 is thus slidable along flats 142 of the drive connector 114.

The tool driver 110 further includes a screw connector 112 that includes a spool 182 to which shaft 128 is connected. The screw connector 112 further includes a bit 178 for cooperation with a slot on the screw 2F. The bit 178 may include a detent 139 for securing the screw to the bit 178. The spool 182 includes flats 140 that mate with internal flats 138 of the collar 132.

The collar 132 cooperates with spring 144 to form actuator 116 for selectively engaging and disengaging the drive connector 114 to the shaft 128. The spring 144 may be slidably fitted over the flats 140 of the spool 182 and restrained by flange 184 and recess 186. Collar 132 may be advanced in the direction of arrow 146 with respect to the spool 182 and the drive connector 114 from first relationship 134 in which the shaft 128 is rotatably connected to the drive connector 114 to a second relationship 136 in which the shaft 128 is rotatably disconnected from the drive connector 114. When the tool driver 110 is in the second relationship 136 the operator may rotate the collar 132 to cause the shaft 128 to similarly rotate while the drive connector 114 may remain stationary.

Referring now to FIG. 30A, yet another embodiment of the present invention is shown as tool driver 11E. The tool driver 110E includes a shaft 128E, which includes a bit 178E. The bit 178E is different than the bit 178 of the tool driver 110 in that the bit 178E has a, for example, polygon cross-section, for example, a hexagonal periphery. The bit 178 may include a detent 139E to help secure the screw.

Referring now to FIG. 31, yet another embodiment of the present invention is shown as tool driver 110F. Tool driver 110F is similar to the tool driver 110 of FIG. 30 except that the tool driver 110F includes a different mechanism for engaging and disengaging the tool driver 110F. The tool driver 110F does not include the internal and external flats 138, 140, 142 of the tool driver 110. Instead, the tool driver 110F includes a pin and collar arrangement.

For example and as shown in FIG. 31, the tool driver 110F includes a drive connector 114F, which includes a collar 188F to which a pin 144F is transversely slidably mounted. The pin 144F is selectably engagable with screw connector 112F through holes 143F formed on the screw connector 112F.

When the pin 144F is disengaged from the holes 143F, the screw connector 112F is free to rotate with respect to the drive connector 114F. When the pin 144F is engaged with the holes 143F of the screw connector 112F, the drive connector 114F is rotatably engaged with the screw connector 112F. A bit 178F extends from the screw connector 112F and is adapted to engage with the screw 2 to tighten the screw 2 into bone 4.

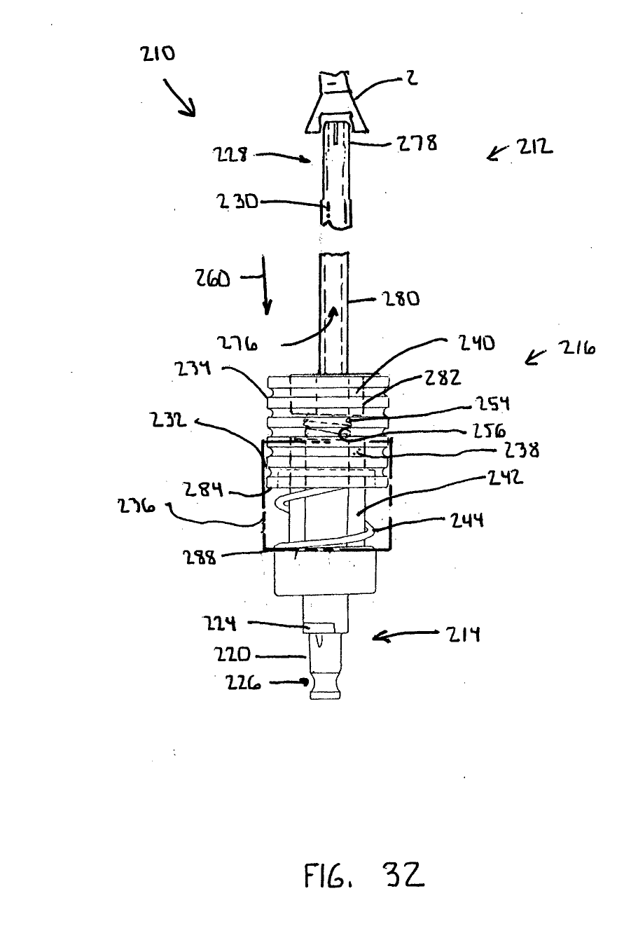

Referring now to FIG. 32, yet another embodiment of the present invention is shown as tool driver 210. Tool driver 210 is similar to the tool driver 10 of FIG. 8 except that the tool driver 210 is adapted so that collar 232 operates in a reverse direction to lock and unlock the tool driver 210. For example and as shown in FIG. 32, the tool driver 210 includes a drive connector 214, an expandable connector 212 and an actuator 216. The drive connector 214 includes a stem 220 and a flange 288 extending from the stem 220. The stem 220 includes flats 224 and groove 226 for rotatably driving the tool driver 210.

The expandable connector 212 includes a flexible member 228. The flexible member 228 includes a spool 282 defining external flats 240 thereon. The flexible member 228 further includes a tube 280, which defines an external periphery 278 thereof for cooperation with screw 2.

The tube 280 defines a longitudinal aperture 276 for slidably receiving flexor 230. The flexor 230 and the flexible member 228 combine and cooperate to form the expandable connector 212.

The external flats 240 on the spool 282 and external flats 242 formed on the drive connector 214 cooperate with internal flats 238 formed on collar 232 to provide for a first relationship 234 (as shown in solid) in which the expandable connector 212 and the drive connector 214 are rotatably connected and a second relationship 236 (as shown in phantom) in which the expandable connector 212 and the drive connector 214 are rotatably independent from each other.

A spring 144 is slidably positioned over the drive connector 214 and constrained between flange 288 and the collar 232 to urge the collar 232 into first relationship 234. The spool 282 includes a flange 284 to constrain the collar 232 within the spool 282. External threads 254 on the drive connector 214 are threadably engaged with internal threads 256 formed in the spool 282.

The collar 232 may be advanced in the direction of arrow 260 to move the collar 232 from first relationship 234 to second relationship 236. When the collar 232 is in the second relationship 236, the drive connector 214 may be rotated relative to the expandable connector 212 to urge the tube 280 in the direction of arrow 260 to cause the flexor 230 to expand the flexible member 228 to expand the external periphery 278 to secure the tool driver 210 to the screw 2.

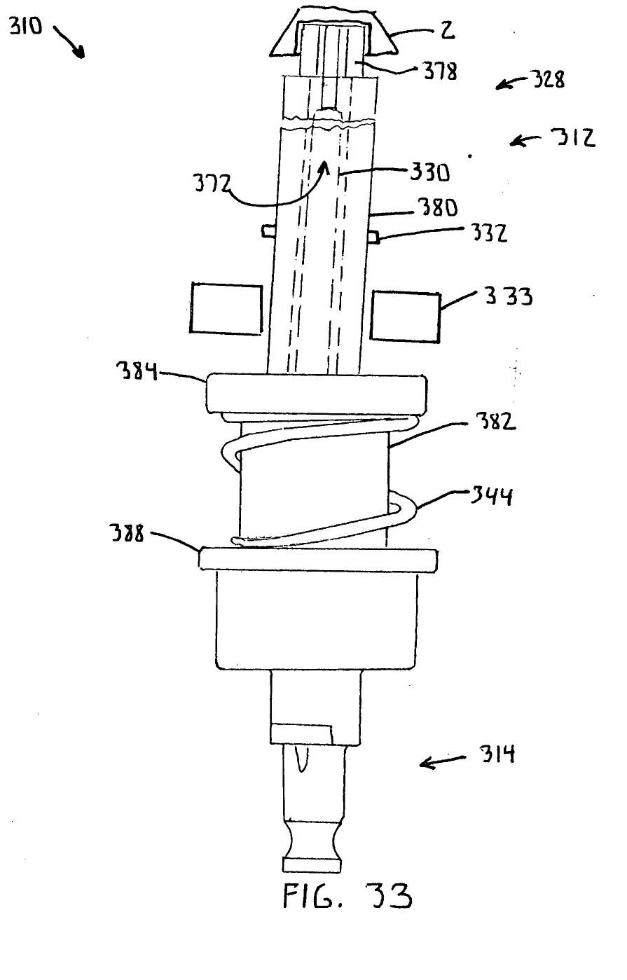

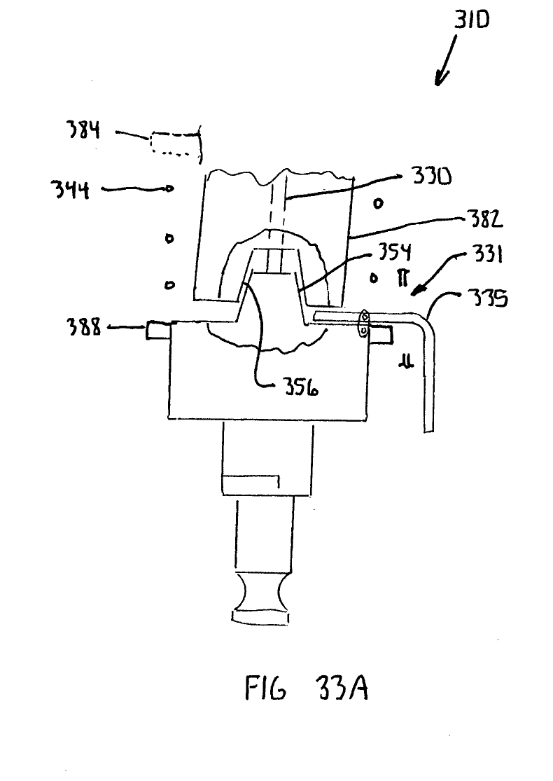

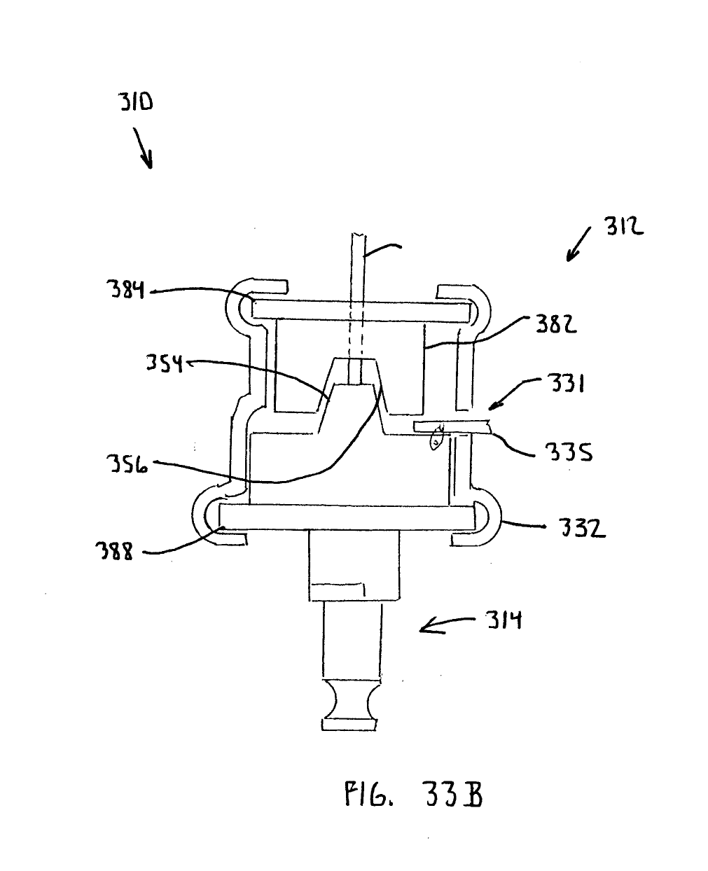

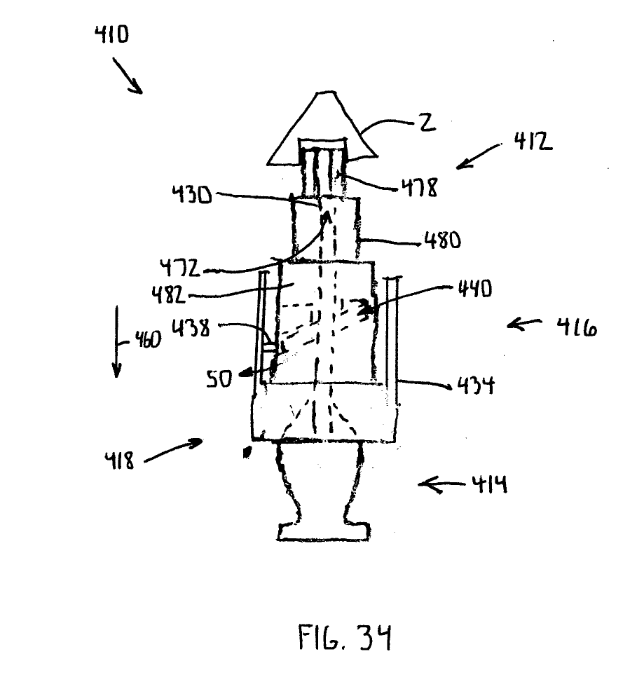

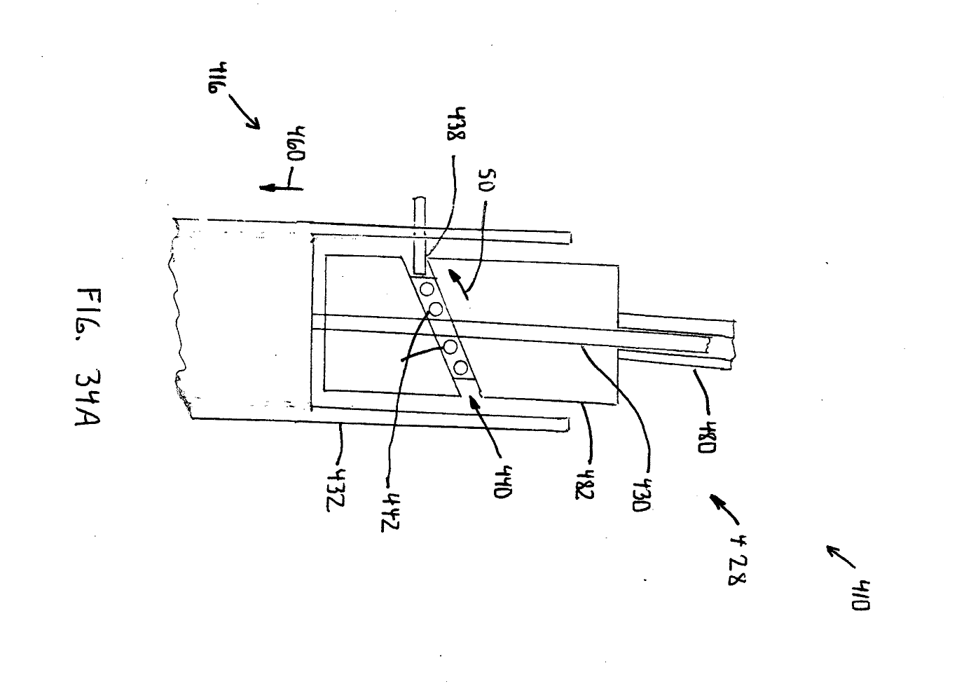

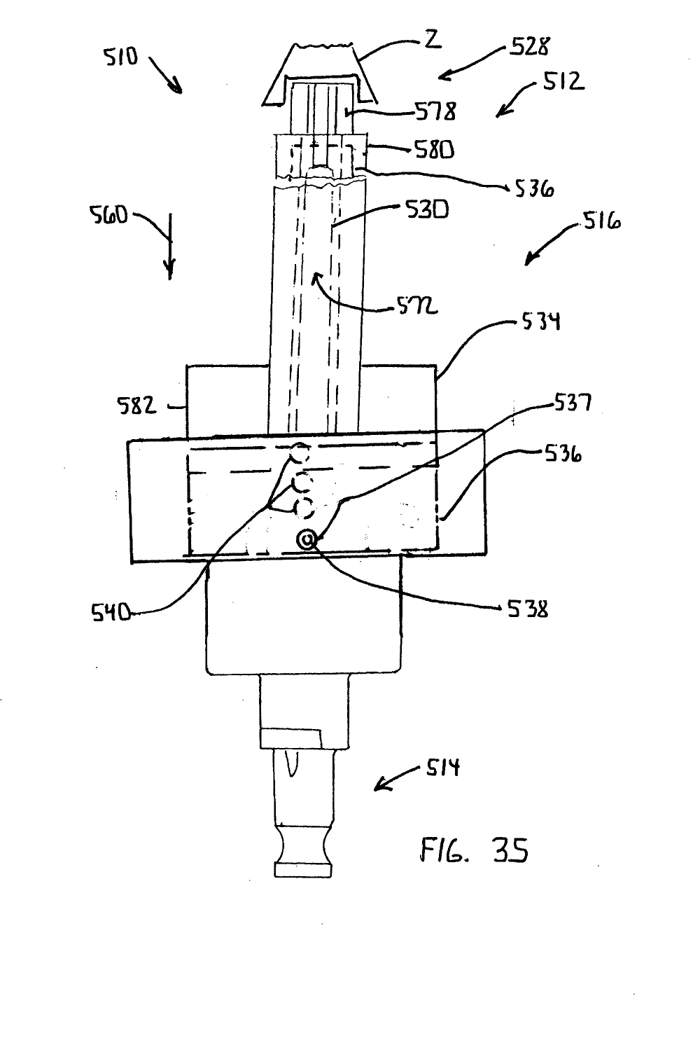

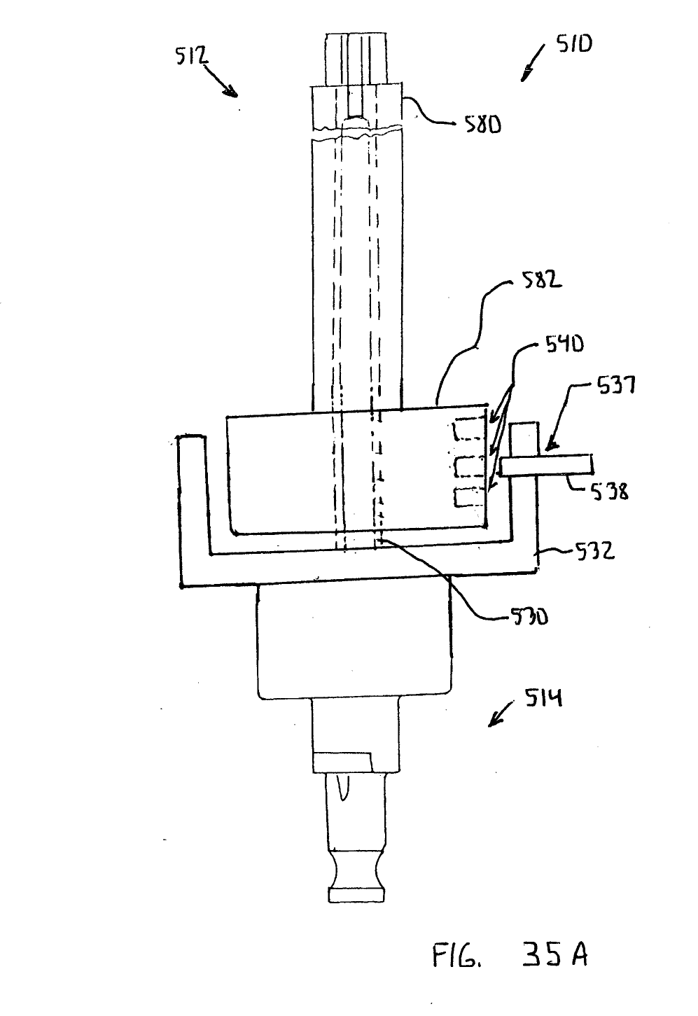

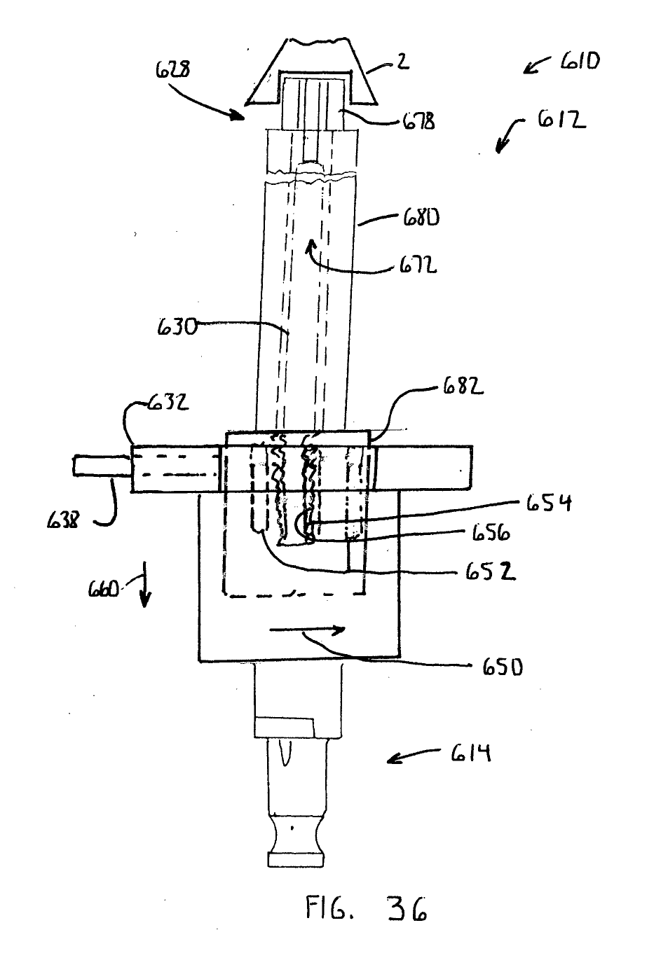

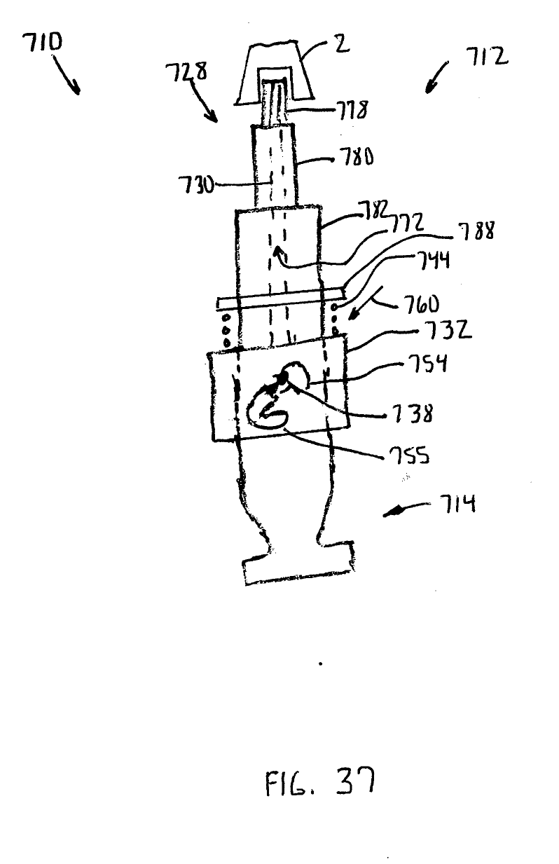

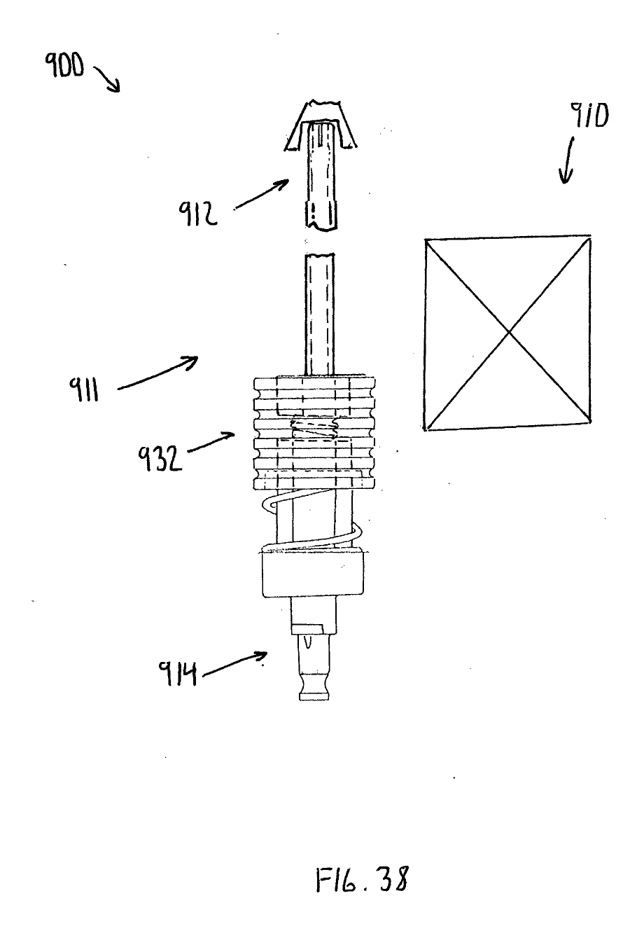

Referring now to FIGS. 33, 33A and 33B, yet another embodiment of the present invention is shown as tool driver 310. The tool driver 310 is similar to the driver 10 of FIG. 8 except that the tool driver 310 utilizes a taper lock engagement rather than a collar with flats to rotatably engage and rotatably disengage the drive connector to the expandable connector.

For example and as shown in FIG. 33B, the tool driver 310 includes a drive connector 314, which is operably connected to an expandable connector 312. A sleeve 332 is positioned between the drive connector 314 and the expandable connector 312 and serves to constrain the drive connector 314 to the expandable connector 312 when they are disengaged from each other.

As shown in FIG. 33, the expandable connector 312 is similar to the expandable connector 12 of the tool driver 10 of FIG. 8. The expandable connector 312 includes a spool portion 382 for selectable connection to the drive connector 314 and a tube portion 380 extending from the spool portion 382. The tube portion 380 and spool portion 382 define a central opening 372 therein. The tube portion 380 further defines an external periphery 378 thereof for cooperation with the screw 2.

The tool driver 310 may as shown in FIG. 33 include an actuation ring 330 slidably positioned over the tube portion 380 and restrained by stop 332 formed on the tube portion 380 and the spool portion 382. Spool portion 382 is selectively matedly connected to the drive connector 314 by a tapered connection.

For example as shown in FIG. 33A, the spool portion 382 includes an internal taper 356 which is selectively engagable with external taper 354 extending from the drive connector 314. A spring 344 may be positioned between flanges 384 and 388, respectively secured to the spool portion 382 and the drive connector 314.

Referring now to FIG. 33B, a releasing arm 335 may be positioned between the spool portion 382 and the drive connector 314 for selectively releasing the tapers 354 and 356 from each other. Releasing arm 335 may be attachable to the drive connector 314 or the spool portion 382 and may be accessed through window 331 in the sleeve 332. A flexor 330 may fit within cavity 372 of the tube 380 and cooperate with the tube portion 380 of flexible member 328 to expand the external periphery 378 to engage with the screw 2.

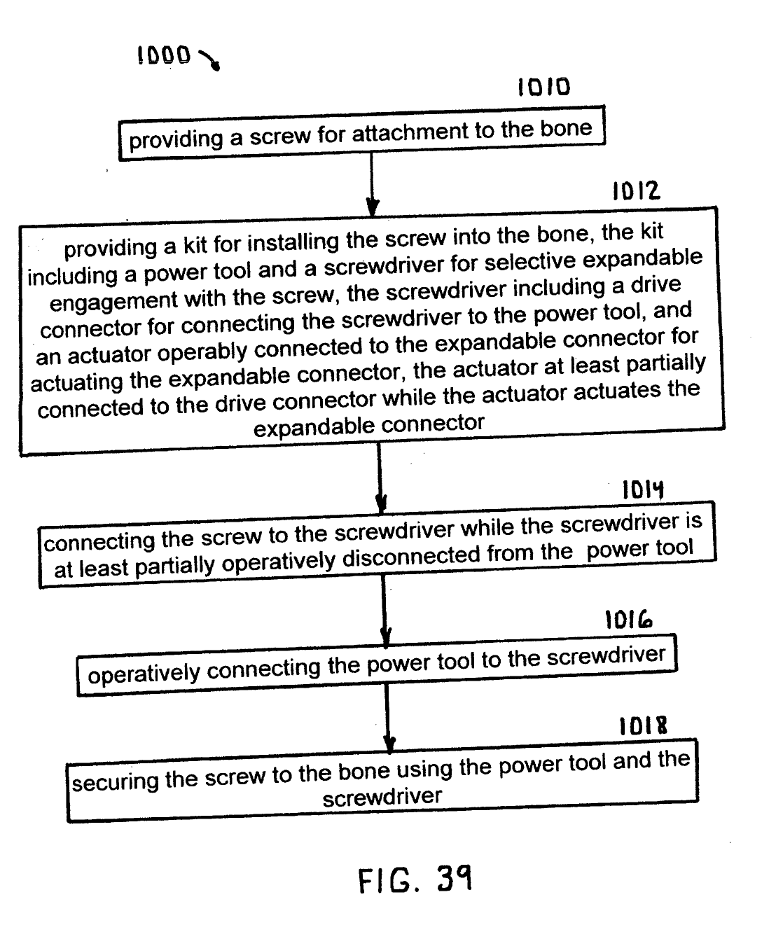

Referring now to FIGS. 34 and 34A yet another embodiment of the present invention is shown as tool driver 410. The tool driver 410 is similar to the tool driver 10 of FIG. 8, except that the tool driver 410 does not use the mating internal and external threads to assist in expanding the flexible member. The tool driver 410 also does not use a series of internal and external flats to selectively engage and disengage the drive connector to the expandable connector.

For example and as shown in FIG. 34, the tool driver 410 includes a drive connector 414 having a drive adapter 418 similar to the drive adapter 18 of the tool driver 10 of FIG. 8. The drive connector 414 further includes a collar 434, which defines a cavity therein for receiving spool portion 482 of flexible member 428.

The tool driver 410 further includes an expandable connector 412, which includes the flexible member 428, which cooperates with flexor 430 to selectively expand periphery 478 of tube portion 480 of the flexible member 428. The expandable connector 412 defines a longitudinal opening 472 for slidably receiving the flexor 430.

Referring now to FIG. 34A, the periphery of the spool portion 482 of the flexible member 428 defines a spiral groove 440 formed thereon. A pin 438 is transversely mounted inwardly from the collar 434 and cooperates with the spiral groove 440 formed on the spool portion 482 to form actuator 416 for actuating the flexible member 428 to secure the screw with the tool driver 410. Holes 442 may be formed in the spool portion 482 in the spiral groove 440. The holes 442 may cooperate with the pin 438 to selectively lock the pin 438 and the drive connector 414 to the spiral groove 440 and the flexible member 428.

Referring now to FIG. 34, as the drive connector 414 is rotated in the direction of arrow 50 with respect to the collar 434, the pin 438 causes the spool portion 482 to advance in the direction of arrow 460 causing the tube 460 to advance in the direction of arrow 480 causing the periphery 478 of the flexible member 428 to expand to secure the tool driver 410 to the screw 2. It should be appreciated, that after the screw 2 is secured to the flexible member 428, the rotation of the drive connector 414 will cause the expandable connector 412 to rotate with the drive connector 414 to further tighten the screw. It should be appreciated that the tool driver 410 is designed for either use with right-hand or left-hand screws.

Referring now to FIG. 35 yet another embodiment of the tool driver of the present invention is shown as tool driver 510. The tool driver 510 is similar to the tool driver 410 of FIG. 34 except the tool driver 510 utilizes a combination of pins and pin holes and does not utilize the spiral groove of the tool driver 410 of FIG. 34.

For example and as shown in FIG. 35, the tool driver 510 includes a drive connector 514 somewhat similar to the drive connector 414 of the tool driver 410 of FIG. 34. The drive connector 514 includes a collar 532 that extends from the connector 514. The collar is slidably fitted over the spool portion 582 of the flexible member 528 of connector 514.

Referring now to FIG. 35A, the collar 532 includes a transverse opening 537, which slidably receives a pin 538. Pin 538 extends through the collar 532 and is retractably cooperable with series of axially spaced apart openings 540 formed in spool portion 582 of the flexible member 528 of expandable connector 512.

The pin 538, opening 537 and openings 540 combine to form the actuator 516 to assist in actuating the flexible member 528 of the expandable connector 512.

Referring again to FIG. 35, the expandable connector 512 includes the spool portion 582, which is slidably fitted through longitudinal opening 572 formed in the spool portion 582 with flexor 530, which is secured to and extends from the drive connector 512. Tube portion 580 of the flexible member 528 of the expandable connector 512 extends from the spool portion 582 and defines an external periphery 578 of the flexible member 528 for cooperation with the screw 2.

As the spool portion 582 is advanced in the direction of arrow 560, the tube portion 580 of the flexible member 528 advances in the direction of arrow 560 and the flexor 530 serves to expand the tube 580 such that the periphery 578 expands to secure the screw 2. Thus, as the spool advances in the direction of arrow 560, the flexible member 528 advances from its first relationship 534 with respect to the drive connector 514 to its second relationship 536 (as shown in phantom).

Referring now to FIG. 36, yet another embodiment of the present invention is shown as tool driver 610. The tool driver 610 is similar to the tool driver 510 of FIG. 35 except that the tool driver 610 further includes an external thread 654 formed on the drive connector 614 which is threadedly engaged with internal threads 656 formed in spool portion 682 of the flexible member 628 of the expandable connector 612.

The tool driver 610 further includes the expandable connector 612, which includes the flexible member 628, which has the spool portion 682 as well as the tubular portion 680. The flexible member 628 includes a central longitudinal opening 672, which receives a flexor or pin 630, which is slidabley receivable therein.

The drive connector 614 includes a transverse opening 632, which slidably receives a pin 638, which is selectively engagable with longitudinal slots 652 formed in the spool portion 682.

As the drive connector 614 is rotated in the direction of arrow 650 with respect to the spool portion 682, the flexible member 628 advances in the direction of arrow 660 causing the flexor 630 to engage with the tubular portion 680 of the flexible member 628 causing the flexible member 628 to expand and the periphery 678 of the flexible member 628 to positively engage the screw 2.

It should be appreciated that as the drive connector 614 continues to advance or be rotated in the direction of arrow 650, the spool portion 682 and, consequently, the flexible member 628 begin to rotate in the direction of arrow 650 causing the tool driver 10 to hand-tighten the screw. After the screw is hand-tightened sufficiently, the pin 638 is advanced centrally to engage the slot 652 to provide for the ability to use the tool driver as a power tool.

It should be appreciated that while it is possible to continue to tighten the screw without the engagement of the pin 638 with a power tool, to do so may cause additional force to be transmitted between the flexor 630 and the flexible member 628 causing potential damage to the tool driver 610.

Referring now to FIG. 37, yet another embodiment of the present invention is shown as tool driver 710. The tool driver 710 is similar to the tool driver 510 of FIG. 35 except that the tool driver 710 provides for a locking feature to lock the flexible member to the screw when in the engaged position.

For example and referring to FIG. 37, the tool driver 710 includes a drive connection 714 which is similar to the drive connection 514 of the tool driver 510 of FIG. 35. The drive connector 714 includes a collar 732, which defines an elongated slot 754 extending obliquely along the periphery of the collar 732. The slot 754 includes a notch 755 extending from the lower end of the slot 754.

The tool driver 710 further includes an expandable connector 712 for securing a screw with the tool driver 710. The expandable connector 712 includes a flexible member 728, which includes a portion having a periphery 778 for cooperation with the screw 2. The periphery 778 is formed on tube portions 780 of the flexible member 728. The flexible member 728 further includes a spool portion 782 extending downwardly from the tube portions 780.

The flexible member 728 includes a central-longitudinal opening 772 extending through the tube portion 780 and the spool portion 782. The spool portion 782 is slidably fitted inside the collar 732 of the drive connector 714. The spool portion 782 is also slidably mounted on pin on flexor 30 extending upwardly from the drive connector 714.

The flexor 730 selectively cooperates with the flexible member 728 to expand the flexible member 728 and thereby enlarge the periphery 778 to engage the screw 2. Spring 744 is slidably positioned over the spool portion 782 and between collar 732 and flange 788 to urge the flexible member 728 and the drive connector 714 in a spaced apart and relaxed position from the periphery 778 to permit the tool driver 710 to be engaged into the screw 2.

To operate the tool driver 710, the spool portion 782 is advanced obliquely in the direction of the arrow 760 such that pin 738 extending through the slot 754 from the spool portion 782 through the collar 732 may advance along the slot 754 in the direction of arrow 760 to the lower portion of the slot 754 where it may then engage with notch 755. The spring 744 then cooperates with the notch 755 and the pin 738 to lock the flexible member 728 into an engaged position with the screw 2.

Referring now to FIG. 38, yet another embodiment of the present invention is shown as ‘kit 900’ for use in orthopaedics in installing a screw to a bone. The kit includes a power tool 910 and a driver 911 for selectively expandable engagement with the screw 2. The driver 911 includes a drive connector 914 for connecting the driver 911 with the power tool 910 and a screw connector 912 for connecting the screw to the driver 911. The driver 911 also includes a coupler 932 for selectively, at least partially operatively, connecting and disconnecting the screwdriver 911 to the drive connector 914.

Referring now to FIG. 39, yet another embodiment of the present invention is shown as surgical procedure 1000. The surgical procedure 1000 is for use in performing orthopaedic surgery on a bone. The method 1000 includes a first step 1010 of providing a screw for attachment to the bone. The method 1000 further includes a second-step 1012 of providing a kit for installing the screw onto the bone. The kit includes a power tool and a screwdriver for selectively expandably engaging the screw. The screwdriver includes a drive connector for connecting the screwdriver to the power tool and a coupler for selectively and at least partially operatively connecting and disconnecting the screwdriver to the drive connector.

The method 1000 further includes a third step 1014 of connecting the screw to the screwdriver while the screwdriver is at least partially operatively disconnected from the power tool. The method 1000 further includes a fourth step 1016 of operatively connecting the power tool to the screwdriver and a fifth step 1018 of securing the screw to the bone using the power tool and the screwdriver.

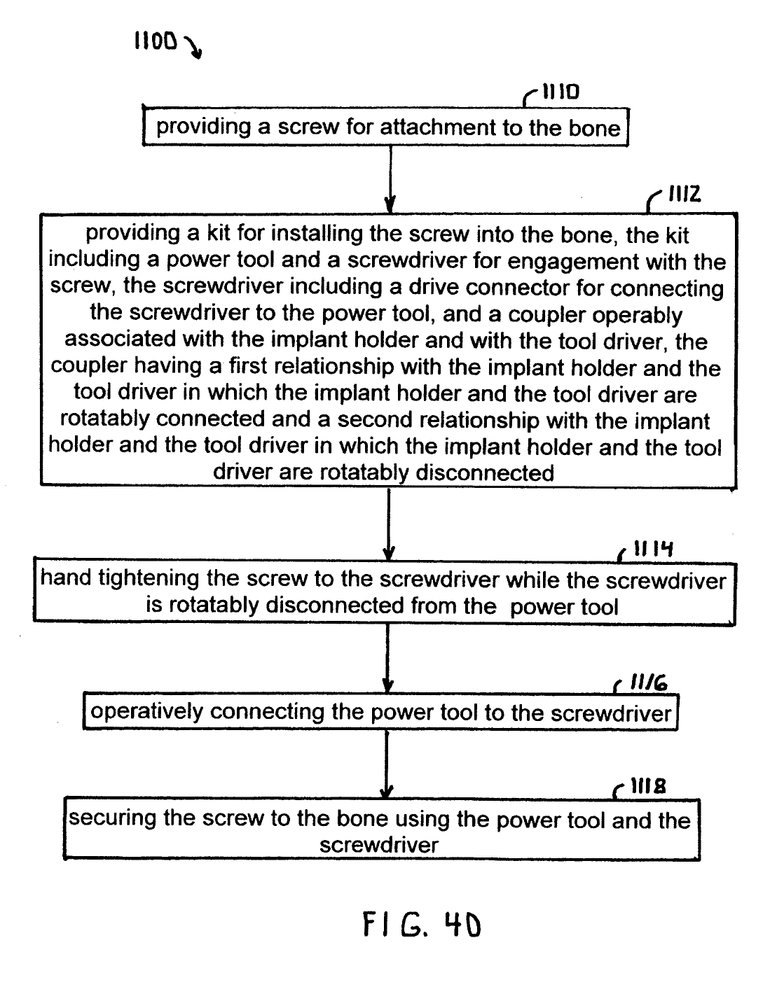

Referring now to FIG. 40, yet another embodiment of the present invention is shown as the surgical procedure 1100 for performing orthopaedic surgery on a bone. The method 1100 includes a first step 1110 of providing a screw for attachment to the bone. The method 1100 includes a second step 1112 of providing a kit for installing the screw onto the bone. The screw includes a power tool and a screwdriver for engagement with the screw. The screwdriver includes a drive connector for connecting the screwdriver to the power tool and a coupler operatively associated with the implant holder and with the implant driver. The coupler has a first-relationship in which the implant holder and the implant driver are rotatably connected and a second relationship with the implant holder and the tool driver in which the implant holder and the tool driver are rotatably disconnected.

The method 1100 further includes a third step 1114 of hand-tightening the screw to the screwdriver while the screwdriver is rotatably disconnected from the power tool and a fourth step 1116 of operably connecting the power tool to the screwdriver. The method further includes a fifth step 1118 of securing the screw to the bone using the power tool and the screwdriver.

There is a plurality of advantages of the subject invention arising from the various features of the subject invention described herein. It will be noted that further alternative embodiments of the subject invention may not include all of the features described yet still benefit from at least some of the advantages of such features. Those of ordinary skill in the art may readily devise their own implementations of the subject invention that incorporate one or more of the features of the subject invention and that fall within the spirit and scope of the subject invention.

What is claimed is:

1. A tool driver for use in orthopaedics to install an implant into bone with a power source, said tool driver comprising:
an expandable connector cooperable with the implant for holding the implant to said expandable connector;
a drive connector for connecting said tool driver to the power source; and
an actuator operably connected to said expandable connector for actuating said expandable connector, said actuator at least partially connected to said drive connector while said actuator actuates said expandable connector.

2. The driver of claim 1, wherein said expandable connector comprises:
a flexible member for contact with the implant; and
A flexor positioned at least partially in intimate contact with said flexible member for flexing said member into one of engagement or disengagement with the implant.

3. The driver of claim 1, wherein said expandable connector comprises a resilient material having at least one slit therein.

4. The driver of claim 1, wherein said expandable connector comprises a collet.

5. The driver of claim 1, wherein said expandable connector comprises:
a hollow tube defining a portion thereof having a slit there through and defining a surface for cooperation with the implant; and
a pin for sliding cooperation at least partially with the hollow tube, said pin cooperating with said tube to expand said tube to secure said tube to the implant.

6. The driver of claim 1, wherein said expandable connector comprises:
a first component cooperable with the implant; and
a second component operably connected to said first component and to said actuator.

7. The driver of claim 6, further comprising a third component cooperable with a second implant having at least one dimension different than said first mentioned implant, said third component operably connected to said second component.

8. The driver of claim 1, wherein said actuator comprises a member operably associated with said expandable connector and to said drive connector, said member having a first relationship with said expandable connector and said drive connector in which said expandable connector and said drive connector are rotatable connected and a second relationship with said expandable connector and said drive connector in which said expandable connector and said drive connector are rotatably disconnected.

9. The driver of claim 8, wherein said member comprises a collar.

10. The driver of claim 8:
wherein said member comprises a collar defining internal flats therein;
wherein said expandable connector defines external flats thereon; and
wherein said drive connector defines external flats thereon.

11. The driver of claim 1, wherein said actuator comprises:
a collar selectable connecting said expandable connector to said drive connector; and
a spring operably connected to said expandable connector and to said drive connector.

12. The driver of claim 1, wherein said actuator comprises:
a first member fixedly attached to one of said drive connector and said expandable connector; and
a second member removably attached to the other of said drive connector and said expandable connector.

13. The driver of claim 12:
wherein said first member comprises a collar fixedly attached to said drive connector; and
wherein said second member comprises a pin removably attached to said expandable connector.

14. A coupler for use with a tool driver and an implant holder for use in orthopaedics to install an implant into bone with a power source, said coupler comprising a member operably associated with the implant holder and with the tool driver, said member having a first relationship with the implant holder and the tool driver in which the implant holder and the tool driver are connected and a second relationship with the implant holder and the tool driver in which the implant holder and the tool driver ate at least partially disconnected.

15. The coupler of claim 14, wherein said member has a first relationship with the implant holder and the tool driver in which the implant holder and the tool driver are rotatable connected and a second relationship with the implant holder and the tool driver in which the implant holder and the tool driver are rotatably disconnected.

16. The coupler of claim 15, wherein said member defines internal flats therein for cooperation with external flats formed on the tool driver and the implant holder.

17. The coupler of claim 15:
wherein said member comprises a collar select ably rotatably connecting the implant holder to the tool driver; and
a spring operably connected to the implant holder and to the collar.

18. The coupler of claim 14:
wherein said first mentioned member is removably rotatably attached to one of the tool driver and the implant holder; and
further comprising a second member fixedly attached to the other of the tool driver and the implant holder.

19. The coupler of claim 18:
wherein said second member is fixedly attached to the implant holder;
wherein said first mentioned member comprises a collar removably attached to the tool driver; and
further comprising a spring operably connected to the tool driver and to said collar for urging said collar into engagement with the tool driver.

20. A method for performing orthopaedic surgery on a bone, comprising the steps of:
providing a screw for attachment to the bone;
providing a kit for installing the screw into the bone, the kit including a power tool and a screwdriver for selective expandable engagement with the screw, the screwdriver including a drive connector for connecting the screwdriver to the power tool, and an actuator operably connected to the expandable connector for actuating the expandable connector, the actuator at least partially connected to the drive connector while the actuator actuates the expandable connector;
connecting the screw to the screwdriver while the screwdriver is at least partially operatively disconnected from the power tool;
operatively connecting the power tool to the screwdriver; and
securing the screw to the bone using the power tool and the screwdriver.
